(19) United States
(12) Patent Application Publication
(10) Pub. No.: US 2001/0044147 A1
(43) Pub. Date: Nov. 22, 2001

(54) Delivery vehicles comprising stable lipid/nucleic acid complexes

(76) Inventors: Sean M. Sullivan, Danville, CA (US); Hans Hofland, San Francisco, CA (US)

Correspondence Address: Roylance, Abrams, Berdo & Goodman, L.L.P., Suite 600, 1300 19th Street, N.W., Washington, DC, 20036, US

(21) Appl. No.: 09/855,796

(22) Filed: May 16, 2001

Related U.S. Application Data
(63) Continuation of application No. 08/652,018, filed on May 21, 1996.
Application No. 08/652,018 is a continuation-in-part of application No. 08/450,142, filed on May 26, 1995, now abandoned.

(51) Int. Cl. A61K 48/00; C12N 15/74; C12N 15/86

(52) U.S. Cl. 435/320.1; 435/456; 514/44

(57) ABSTRACT

Stable polynucleotide delivery vehicles (SPDVs) are described which incorporate a polynucleotide/cationic lipid complex as structural components of the SPDV. The subject SPDVs may optionally incorporate synthetic biodegradable amphipathic lipids, and suitable targeting agents.

### RELATED APPLICATION

This application is a continuation-in-part of U.S. application Ser. No. 08/450,142, filed May 26, 1995.

### 1.0 INTRODUCTION

The present invention is in the field of biochemistry. In particular, novel compositions are reported which efficiently deliver polynucleotides or other bioactive materials to cells.

### 2.0 BACKGROUND

The present invention relates to novel polynucleotide delivery vehicles, and novel methods for producing the same.

As the field of molecular biology has matured, a wide variety of methods and techniques have evolved which allow researchers to engineer polynucleotides. Polynucleotides are typically engineered with the goal that they perform a specific function within the cell. Unfortunately, polynucleotide polymers are highly charged molecules (due to the phosphate backbone) and do not readily permeate the cell membrane. As such, concomitant with the advances made in genetic engineering, advances have also been made in methods by which researchers may introduce genetically engineered material into cells.

One of the methods developed for delivering genetically engineered polynucleotides to cells involves the use of liposomes. The phospholipid bilayer of the liposome is typically made of materials similar to the components of the cell membrane. Thus, polynucleotides associated with liposomes (either externally or internally) may be delivered to the cell when the liposomal envelope fuses with the cell membrane. More typically, the liposome will be endocytosed into the cell. After internalization, the internal pH of the endocytic vesicle may drop substantially, and/or the vesicle may fuse with other intracellular vesicles, including lysosomes. During or subsequent to the process of vesicle fusion, the internal contents of the endosome may be released into the cell.

Liposomes are limited as polynucleotide delivery vehicles by their relatively small internal volume of the liposome. Thus, it is difficult to effectively entrap a large concentration of polynucleotide within a liposomal formulation.

Researchers have tried to compensate for the above inefficiency by adding or using positively charged amphipathic lipid moieties to the liposomal formulations. In principle, the positively charged groups of the amphipathic lipids ion-pair with the negatively charged polynucleotides and increase the extent of association between the polynucleotides and the lipidic particles which presumably promotes binding of the nucleic acid to the cell membrane. For example, several cationic lipid products are currently available which are useful for the introduction of nucleic acid into the cell. Particularly of interest are, LIPOFECTIN (DOTMA) which consists of a monocationic choline head group which is attached to diacylglycerol (see generally, U.S. Pat. No. 5,208.036 to Epstein et al.); TRANSFECTAM (DOGS) a synthetic cationic lipid with lipospermine head groups (Promega, Madison, Wis.); DMRIE and DMRIE*HP (Vical, La Jolla, Calif.); DOTAP (Boehringer Mannheim (Indianapolis, Ind.), and Lipofectamine (DOSPA) (Life Technology, Inc., Gaithersburg, Md.).

Properly employed, the above compounds apparently enhance the permeability of nucleic acids to cells cultured in vitro. Accordingly, the process of lipofection has become an important tool of cellular biology. Typically, formulations comprising the cationic lipids are intermixed with the polynucleotide to be delivered and then applied to the target cells. The cationic lipid-polynucleotide complex must generally be used relatively soon after mixing because after a few hours, lipofection efficiency degrades markedly. From this observation, one may surmise that, at least with respect to lipofection efficiency, the cationic lipid-polynucleotide complex is rather unstable.

From a research perspective, the above complexes are rather facile to prepare. Thus, the relatively short active-life of the prepared complex is not an issue where in vitro applications are involved. However, where the medical or in vivo use of polynucleotide delivery vehicles comprising cationic lipids is contemplated, one may not assume that a given clinician would necessarily be capable of reliably preparing an active formulation, and subsequently using that formulation within the rather narrow window of optimum activity. Thus, particularly where clinical use is contemplated, a more stable polynucleotide delivery system would be preferred.

Another draw-back of the presently available compounds is that the respective lipid and cationic components are not joined by a biodegradable chemical linkage. As such, most of the presently available synthetic cationic lipids have proven to be significantly toxic because the target cells cannot metabolize the synthetic lipids.

A given level of cellular toxicity may be detrimental but acceptable where in vitro or research use of cationic lipids to deliver polynucleotides is contemplated; however, such toxicity is generally unacceptable where in vivo use of cationic lipids is contemplated. Thus, cationic lipids which comprise biocompatible, biodegradable, or metabolizable components would be preferred, if not essential, for the preparation of cationic lipid-polynucleotide delivery vehicles for use in vivo. Alternatively, cationic lipid-polynucleotide delivery vehicles of substantially reduced toxicity may be employed.

Finally, the currently available methods for using synthetic cationic lipids to transfect cells all produce lipid/DNA complexes which are rapidly inactivated by relatively low concentrations of serum. Serum sensitivity may be easily circumvented in in vitro applications by conducting the initial portions of the transfection procedure in serum free medium. However, serum sensitivity remains a major obstacle to the wide-spread use of cationic lipid-mediated DNA delivery in vivo.

### 3.0 SUMMARY OF THE INVENTION

The present invention contemplates a novel stable polynucleotide delivering vehicle which retains transfection efficiency for at least 48 hours after formation/synthesis.

Accordingly, the present invention also claims methods of making stable polynucleotide delivery vehicles which comprise: contacting the polynucleotide to be delivered with an amphipathic cationic lipid conjugate while in the presence of detergent; and removing the detergent whereby substantially size stable polynucleotide delivery vehicles are formed which are also substantially stable with respect to transfection efficiency.

Another embodiment of the claimed invention is a stable complex produced as described above with the added feature that the nucleic acid is complexed with a cation prior to, or concurrent with, the addition of detergent, and the detergent is removed prior to the removal of the cation.

Another aspect of the present invention is a process for making a stable polynucleotide delivery vehicle comprising the steps of contacting polynucleotide with cationic lipid in the presence of detergent, removing the detergent to complex the polynucleotide to the cationic lipid, and isolating the resulting delivery vehicles.

The isolated delivery vehicles may be resuspended in a lesser volume than the volume in which they were originally formed. The result being the formation of concentrated compositions comprising delivery vehicles. Thus, another aspect of the present invention is a stable polynucleotide delivery vehicle generally comprising a DNA concentration of at least about 0.5 mg per ml, and preferably at least about 1.0 mg per ml.

During isolation of the stable polynucleotide delivery vehicles, the toxicity of the resulting composition may be drastically reduced. Thus, yet another embodiment of the present invention is a stable polynucleotide delivery vehicle of substantially reduced toxicity.

The present invention further contemplates stable polynucleotide delivery vehicles which comprise amphipathic cationic lipid conjugates which are additionally complexed with noncationic lipids or other lipid moieties.

A further embodiment of the present invention is a stable polynucleotide delivery vehicle which comprises a biocompatible cationic lipid conjugate, and methods for producing and using the same.

As such, the present invention also contemplates biocompatible amphipathic cationic lipid conjugates which comprise a biodegradable lipid moiety which is covalently attached to a biocompatible cationic or polycationic moiety by a pH sensitive chemical linkage which is also biocompatible.

Accordingly, an additional embodiment of the subject invention involves biocompatible amphipathic cationic lipid conjugates having the general formula:

R₁-X-R₂

wherein R₁ is a biodegradable lipid moiety; R₂ is a biocompatible cationic or polycationic moiety; and X is a biocompatible biodegradable or otherwise labile covalent linker.

Because of the stability of the presently contemplated polynucleotide delivery vehicles, targeting groups may be additionally incorporated into the vehicle whereby the polynucleotides to be delivered may be targeted to particular cell types and/or cellular locales (e.g., the nucleus).

Another embodiment of the present invention contemplates the use of the above stable polynucleotide delivery vehicles to deliver a polynucleotide, or polynucleotides, of interest to a cell. In a related aspect, the stable polynucleotide delivery vehicles may be used to provide a therapeutic benefit to the individual.

Yet another aspect of the invention is a method of targeting the stable complexes (or delivery vehicles) to particular cells and tissues by associating the complexes with a targeting agent having the property of being capable of binding the stable complex.

### 4.0 DESCRIPTION OF THE FIGS.

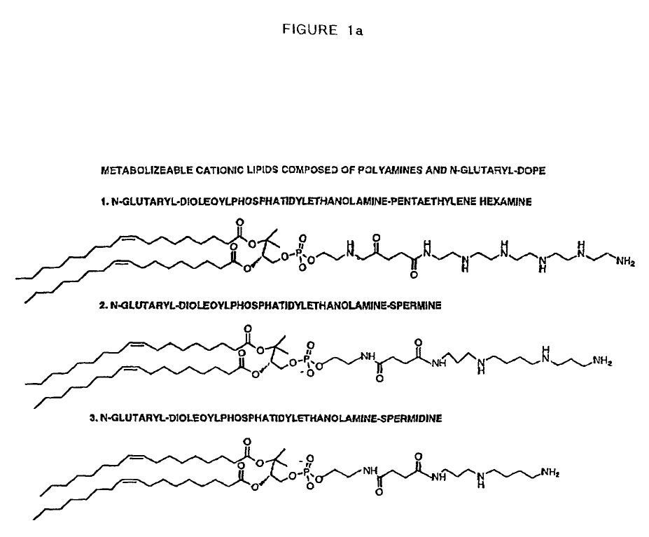

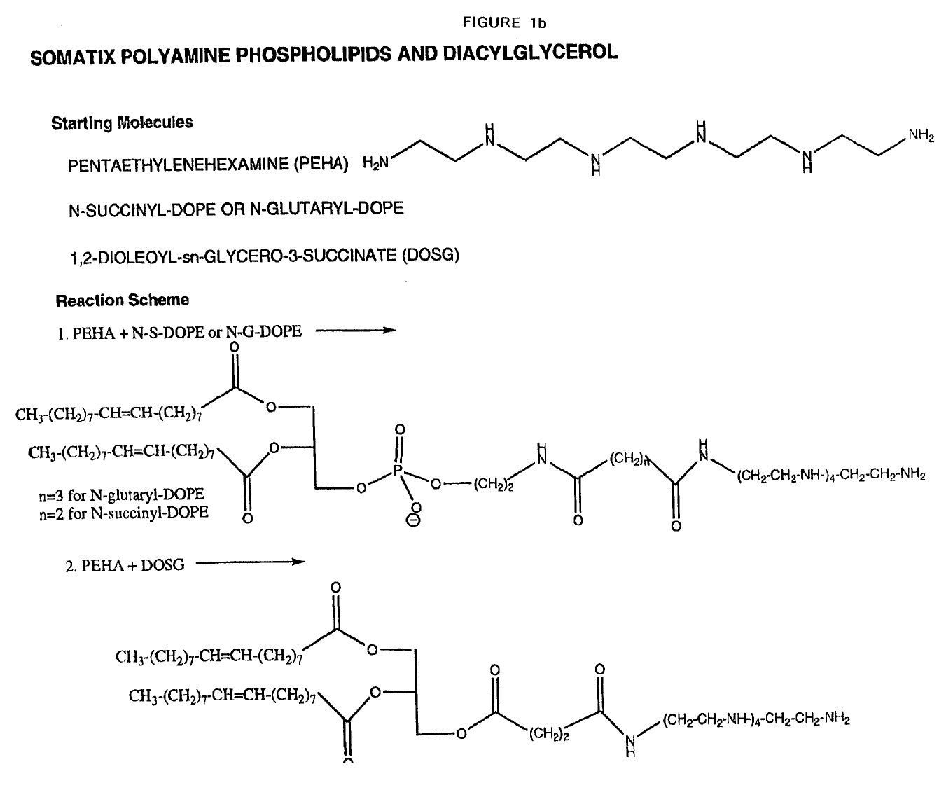

FIGS. 1a and 1b. Show several examples of the novel metabolizable/biodegradable cationic lipids of the present invention. Specifically, examples of N-glutaryl-dioleoylphosphatidylethanolamine conjugated (by a phosphodiester linkage) to hexamine, spermine, spermidine, pentaethylenehexamine (PEHA); N-succinyl-dioleoylphosphatidylethanolamine conjugated to pentaethylenehexamine (by a phosphodiester linkage); 1,2-dioleyl-sn-glycero-3-succinate (DOSG) conjugated to pentaethylenehexamine (by an ester linkage).

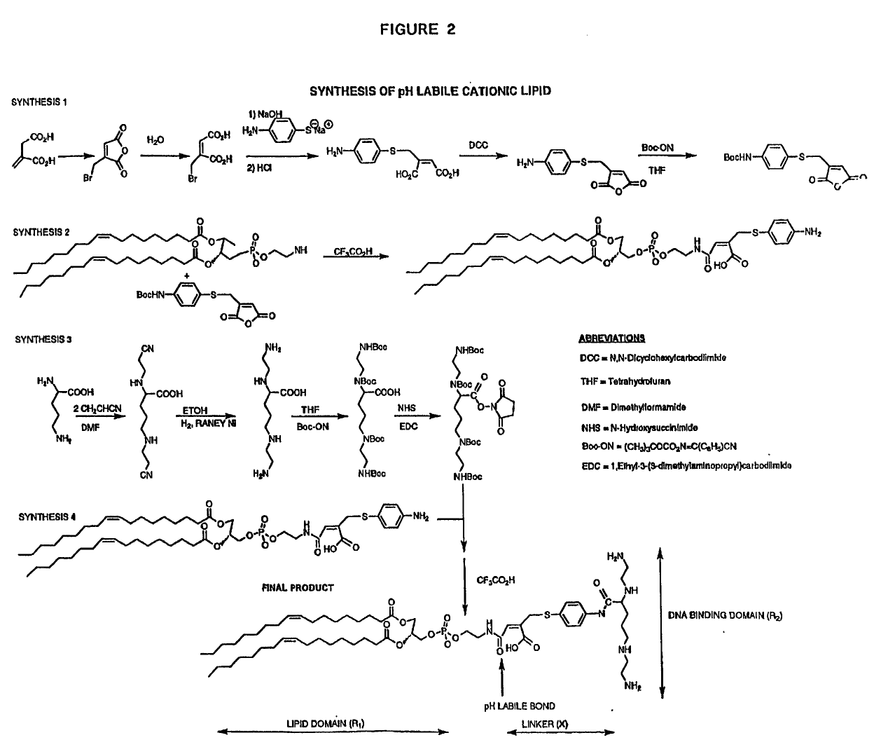

FIG. 2. Shows an example of the novel biodegradable cationic lipids contemplated by the present invention which incorporate a pH labile linker molecule. A reaction scheme for synthesizing the molecule is also provided.

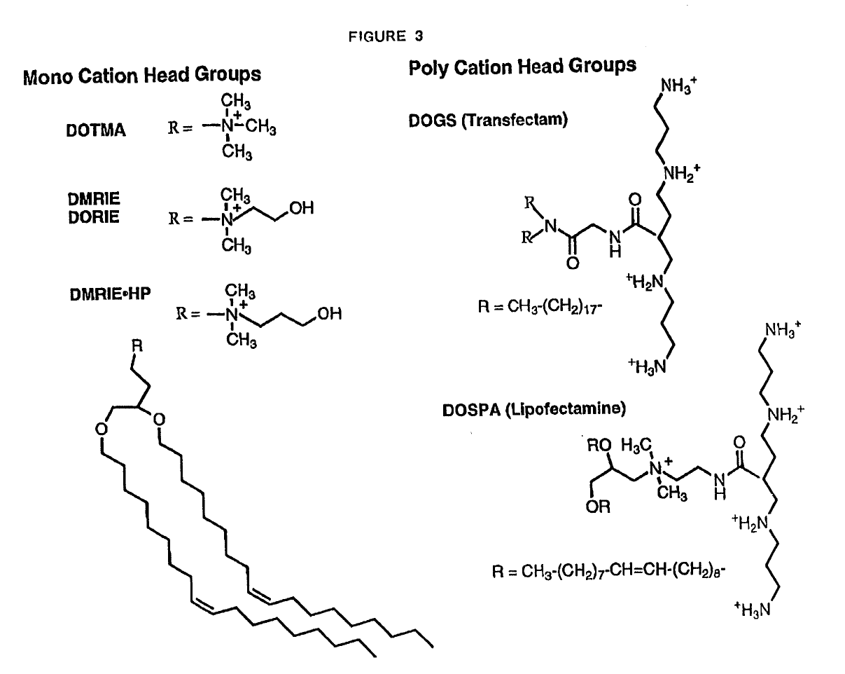

FIG. 3. Shows how and where the cationic groups of some of the presently available cationic lipids are attached to the acyl chains. Specifically, the monocationic synthetic lipids DOTMA, DMRIE, DORIE, and DMRIE.HP; and the polycationic synthetic lipids DOGS (Transfectam™) and DOSPA (Lipofectamine) are shown.

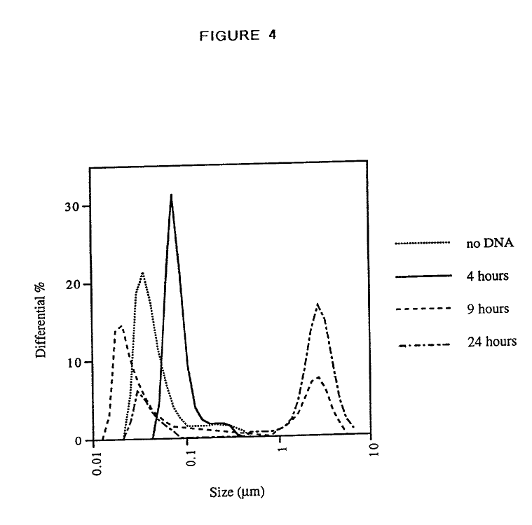

FIG. 4. Shows the change in the size distribution of transient liposome-DNA complexes at several different time points.

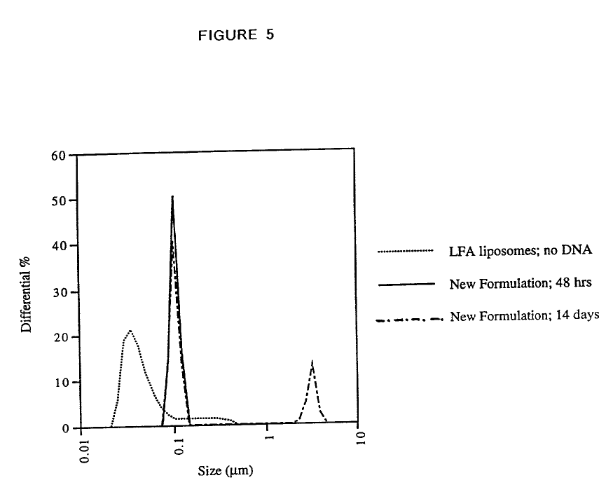

FIG. 5. Shows the change in the size distribution of stable polynucleotide delivery vehicles at several different time points.

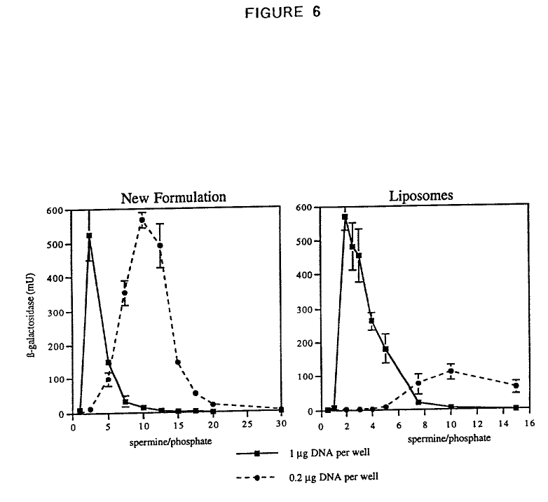

FIG. 6. Shows the relative transfection efficiencies (as measured by beta-galactosidase activity) of transient liposome-DNA complexes, and stable polynucleotide delivery vehicles as a function of lipid/DNA phosphate ratio and the amount of input DNA used to form the complex/vehicle.

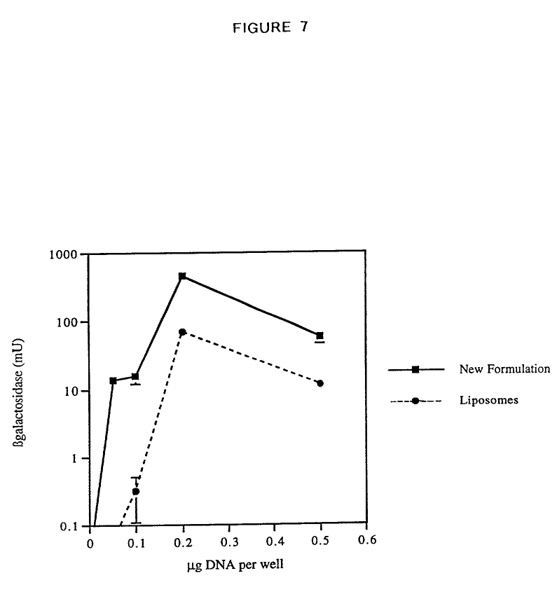

FIG. 7. Shows a more discriminating analysis of the relative transfection efficiencies (as measured by beta-galactosidase activity) of transient liposome-DNA complexes, and stable polynucleotide delivery vehicles as a function of the amount of input DNA used to form the complex/vehicle.

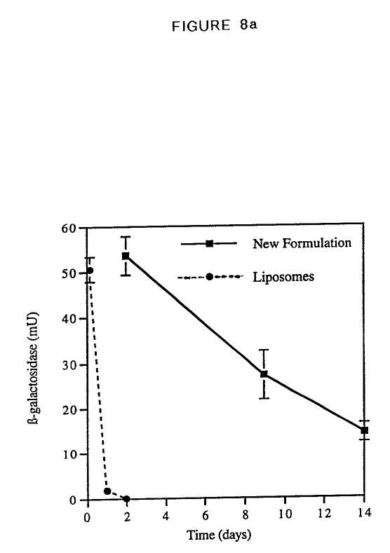

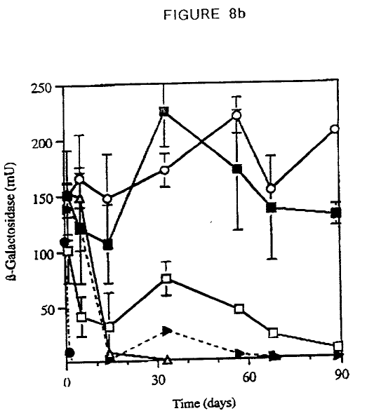

FIG. 8. FIG. 8(a) shows the relative stability (as measured by degradation of transfection efficiency/beta-galactosidase activity) of transient liposome-DNA complexes, and stable polynucleotide delivery vehicles as a function of storage time. FIG. 8(b) shows the relative stabilities of the stable and transient complex under different storage conditions. The stable cationic lipid/DNA complexes were stored at minus 20° C. with five percent dextrose (solid squares), minus 20° C. without dextrose (open squares), 4° C. (open circles), room temperature (solid triangles), and 37° C. (open triangles). The transient complex was stored at 4° C. (solid circles).

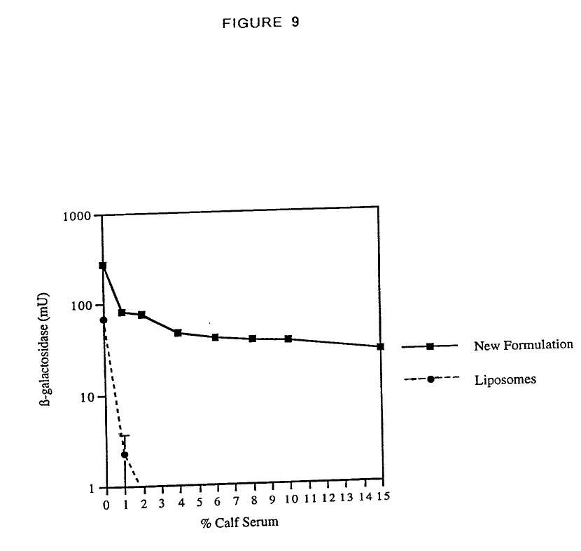

FIG. 9. Shows the relative stability (as measured by degradation of transfection efficiency/beta-galactosidase activity) of transient liposome-DNA complexes, and stable polynucleotide delivery vehicles as a function of percent serum concentration.

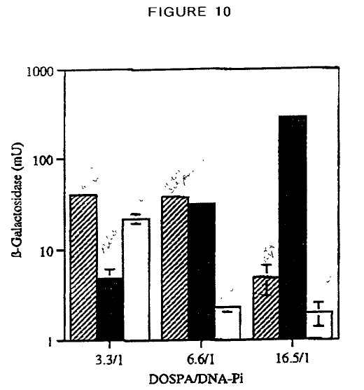

FIG. 10. Shows the gene transfer activity of stable cationic lipid/DNA complex after separation from uncomplexed lipid. Stable cationic lipid/DNA complexes were prepared DOSPA/DNA phosphate ratios of 3.3:1, 6.6:1, and 16.5:1. The suspensions were centrifuged and the pellet (solid black box), the supernatant (open box), and the original uncentrifuged suspension (cross-slashed box) were assayed for gene transfer activity. Approximately 0.2 μg DNA from the original suspension was added to 10⁵ NIH 3T3 cells. Corresponding amounts of pellet and supernatant were added to the cells as based upon providing an equivalent volume of the original suspension prior to centrifugation.

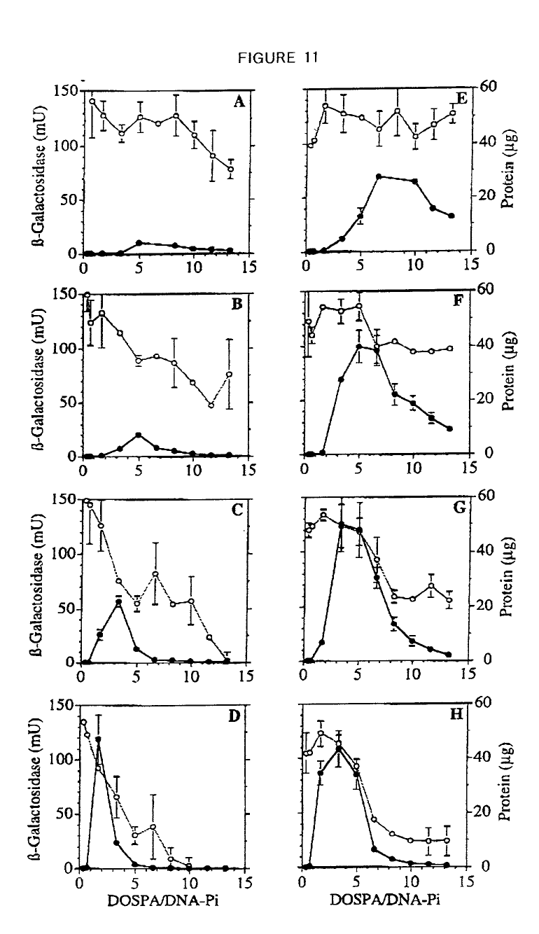

FIG. 11. Is a graph of the DNA dose used to transfect cells as a function of both β-gal activity and total cell protein.

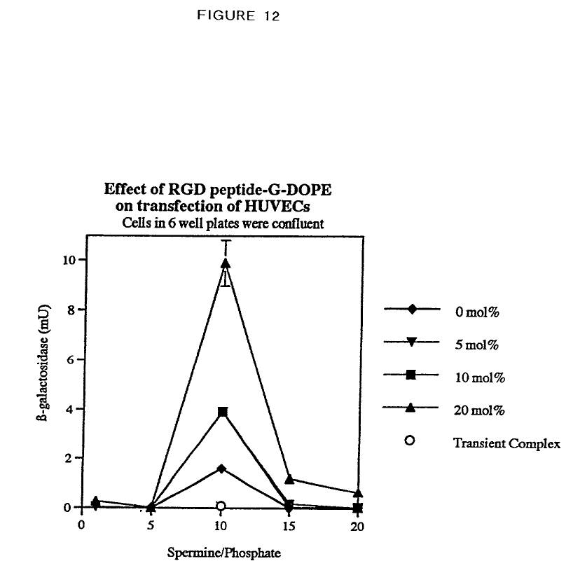

FIG. 12. Shows the comparative levels of expression obtained in targeting studies using an RGD peptide-associated lipid which was incorporated into either transient or stable lipid/DNA complexes at a several different spermine/DNA phosphate ratios, and several different mole percentages of lipid-associated ligands/total lipid.

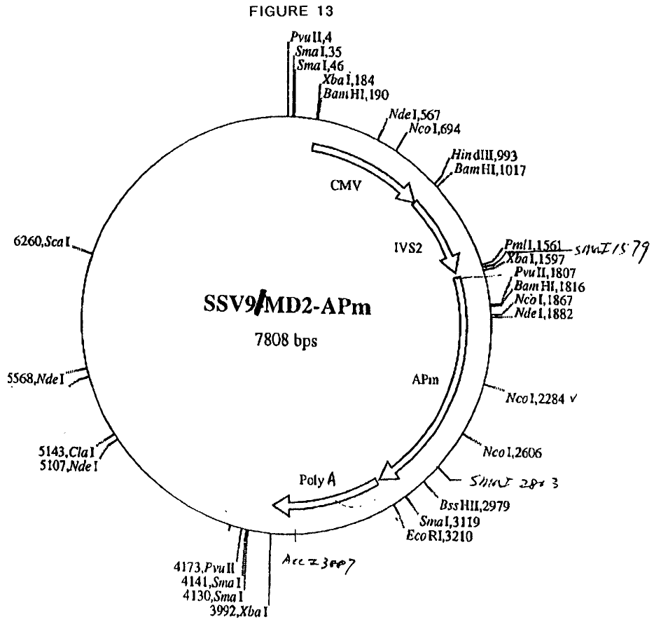

FIG. 13. Shows the effect of DOSPA/DNA phosphate ratio on in vivo gene transfer, and biodistribution using stable synthetic DNA delivery vehicles that were produced in the presence of cation (MnSV101).

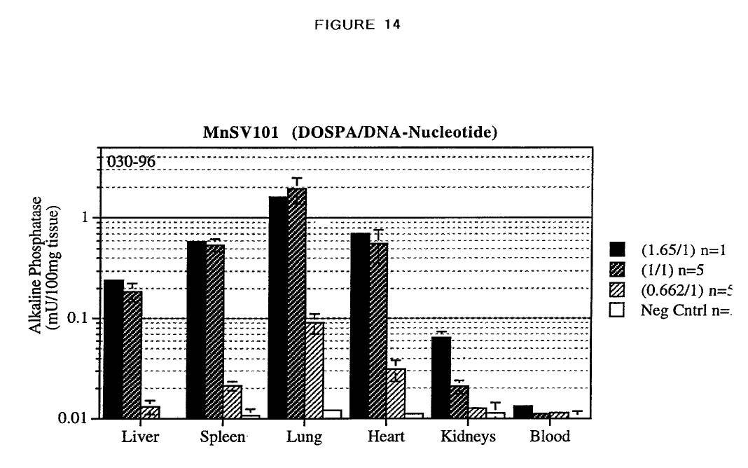

FIG. 14. Shows a dose response curve for biodistribution of MnSV101 (as measured by alkaline phosphatase activity) at a DOSPA/DNA nucleotide ratio of 1:1.

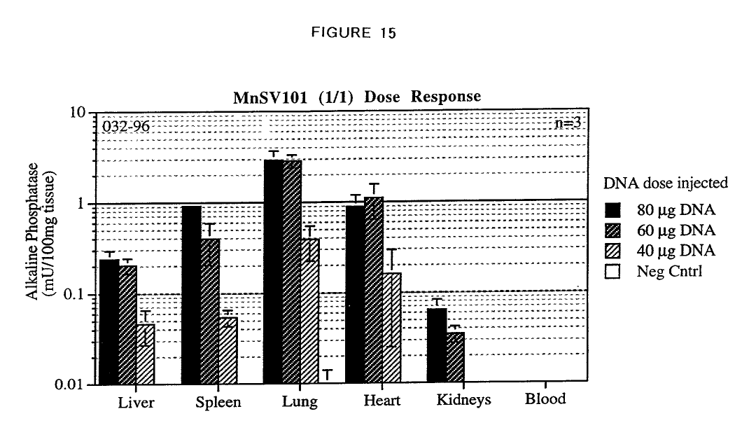

FIG. 15. Shows a time course for in vivo gene transfer and expression by MnSV101 prepared at a DOSPA/DNA nucleotide ratio of 1/1. Alkaline phosphatase expression for each tissue was assayed at designated days by immunocapture.

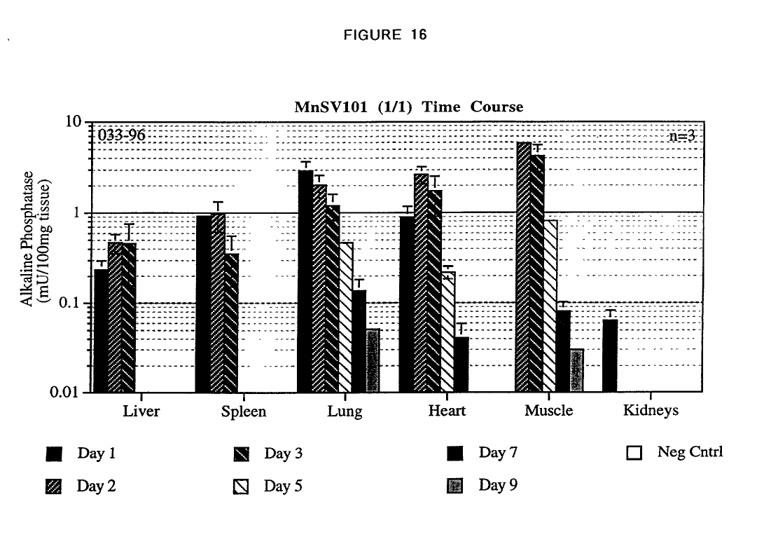

FIG. 16. Shows a comparison between MnSV101 and NaSV101 (formed in the presence of the cation Na instead of Mn) with regards to biodistribution and the efficiency of gene transfer. The DOSPA/DNA nucleotide ratio was kept at 1/1, the injection volume was 0.25 ml, and the DNA dose used was 80 ug. Alkaline phosphatase expression for each tissue was assayed by immunocapture. MnSV101 and NaSV101 were either dialyzed against dextrose in the second dialysis step or saline (0.15M NaCl) prior to injection.

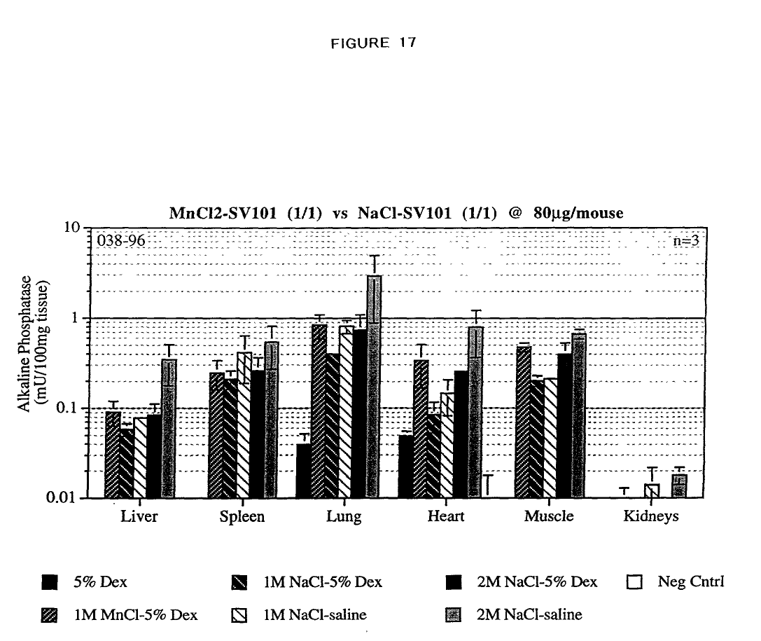

FIG. 17 shows that stable delivery vehicles made using NaCl as a cation are capable of delivering genes to mammalian cells in vivo.

### 5.0 DETAILED DESCRIPTION OF THE INVENTION

The biodegradable amphipathic cationic lipids of the present invention may be contacted (ion paired) with a polynucleotide, or polynucleotides, of interest such that the positive charge of the cationic lipid electrostatically interacts with the negatively charged polynucleotide. The electrostatic interaction between the cationic moiety and the polynucleotide presumably reduces charge repulsion in the polynucleotide and allows the polynucleotide to condense into a more compact configuration (as seen by gel-shift assays).

The condensed cationic lipid/polynucleotide complex subsequently serves as a scaffold or nucleus for the assembly of the polynucleotide delivery vehicle. By physically incorporating the condensed polynucleotide as an integral portion of the structure, the presently described polynucleotide delivery vehicles may stably comprise a more significant proportion of polynucleotide relative to that typically obtained using prior formulations/methods. For example, using the presently disclosed methods, at least about eighty (80) percent of the input polynucleotide remains stably associated with the delivery vehicles of a discrete size-range when measured 48 hours after complex formation.

Preferably, the biodegradable amphipathic cationic lipid conjugates of the present invention comprise biodegradable components. As such, the lipid moiety may comprise any of a number of fatty acids chains (saturated or cis/trans unsaturated) generally having hydrocarbon chains comprising between about 3 to about 26, and preferably between about 12 to about 24 carbon atoms, cholesterol, and derivatives and variations thereof, as long as the lipids are biodegradable or biocompatible.

The cationic component of the present invention may be monovalent, divalent, or preferably polyvalent (i.e., polycationic). The cationic moiety is preferably biocompatible and may comprise any of a variety of chemical groups which retain a positive charge at or near neutral pH including, but not limited to amino groups, amide groups, amidine groups, positively charged amino acids (e.g., lysine, arginine, and histidine), spermine, spermidine, imidazole groups, guanidinium groups, or derivatives thereof.

The cationic component will generally be combined with the polynucleotide at a cation/phosphate ratio that has been optimized for a given application. Usually, the cation/phosphate ratio will be between about 1 and about 20, often between about 5 and about 17, and preferably between about 6 and about 15. The charge ratio will vary accordingly depending on the number of positively charged groups contained on the cation, and the size of the polynucleotide.

Typically, the cationic and lipid components of the claimed biodegradable amphipathic cationic lipid conjugates are described in, or may be obtained from any of a variety of sources including, but not limited to, the 1995 edition of the Merck Index, Budavari, et al., eds., Merck and Company, Inc, Rahway, N.J., the 1995 SIGMA chemical company catalogue, St. Louis, Mo., the 1995 Aldrich Biochemicals Catalogue, or the 1995 Ofatlz and Bauer catalogue.

The cationic group may preferably be attached to the lipid component by an ester or phosphodiester bond which renders the fatty acid separable from the cationic group by the action of natural enzymes such lipases or phospholipases, and the like (see FIG. 1). Such a linkage represents an improvement over the currently available synthetic cationic lipids which attach the lipid using an ether bond which presumably contributes to the cellular toxicity associated with the currently available cationic lipids.

For example, FIG. 1 depicts the chemical structures for polyamines covalently bonded to dioleoylphosphatidyl-ethanolamine (DOPE) using a glutaryl linker. The DOPE can be degraded by phospholipases A₁, A₂, C, and D. This offers advantages over existing synthetic cationic lipids which use ether bonds for attaching the acyl chains. The ether linkages may not be degraded by phospholipases, and thus the ether linked acyl groups accumulate in the cell membrane.

DOSPA, the cationic lipid in Lipofectamine (Life Technology Inc., Gaithersburg, Md.) and DOGS, the cationic lipid for transfectam (Promega) contain spermine attached to a diacylether-linked glycerol. DOSPA and DOGS are theoretically biodegradable because they contain a peptide bond; however, no corroborating data have been presented in the literature which support this notion. Additionally, even if limited hydrolysis were to occur, the resulting degradation product would still be an ether linked diacylglycerol.

Another advantage of the presently disclosed biodegradable amphipathic lipids is the way in which the polyamine is attached to the lipid. The diacyl ether linked glycerol for DOSPA and DOGS are attached to the middle of the spermine. The new molecules are attached at the end of the molecule via an amide bond. FIG. 3 shows the general formulations for many of the monocation and tetracation lipids which are presently available.

In a particularly preferred embodiment, the cationic and lipid moieties of the claimed biodegradable amphipathic cationic lipid conjugates are covalently linked by a labile (e.g., biodegradable or pH labile) linker group. Labile linkers allow for the production of polynucleotide delivery vehicles comprising cationic lipids which dissociate the lipid and cation moieties after cellular internalization and/or endosomal fusion.

The lipid analog may be engineered such that the lipid product can destabilize or disrupt the endosomal membrane to facilitate the release of the cation/nucleotide complex into the cytoplasm. The lipid hydrolysis product may be a diacylglycerol, lys-phosphoryl or phosphatidyl ethanolamine, monoacylglycerol, triglyceride, or the like.

One embodiment of the present invention is a pH labile linker molecule. This linkage is based on 2-methylmaleic anhydride which forms an acid labile link upon reaction with amino groups. As such, pH labile bonds modified as described above serve as working exemplifications of the claimed pH sensitive/labile covalent linker moieties (which may also include ester linkages).

For the purposes of the present invention, the term amphipathic shall refer to a molecule or compound which comprises at least one substantially polar (i.e., freely miscible in aqueous solvent) region and at least one substantially nonpolar (i.e., freely miscible in organic solvent) region. The term biodegradable cationic lipid shall refer to the fact that upon entering into the cell the cationic lipid is converted from an amphipathic molecule to its separate hydrophilic and hydrophobic components (and metabolizable byproducts thereof), or is otherwise capable of participating in the catabolic or metabolic processes of the cell. The term biocompatible shall mean that the compound does not display significant toxicity or adverse immunological effects at the contemplated dosages. The term pH sensitive shall mean that at least one covalent bond in the molecule may be broken by a change in pH that generally approximates that which occurs after endosomal fusion. The term substantially toxic shall mean that, at therapeutic dosages, a given agent produces harmful consequences which, on balance, clearly outweigh the contemplated therapeutic benefits of the agent.

Another method of biodegradably linking the spermine, or other cation, to the lipid involves using dipeptide linkers which are susceptible to proteolytic cleavage by lysosomal proteases, including, but not limited to, thioproteases or cathepsins.

Another embodiment of the present invention are novel methods of using the above-described biodegradable amphipathic cationic lipid conjugates, or currently available cationic lipid conjugates (e.g., Lipofectin, Lipofectamine, and the like) to assemble polynucleotide delivery vehicles which remain stable (in terms of maintaining size and transfection efficiency) under a variety of storage and use conditions.

The stable synthetic polynucleotide delivery vehicles (SPDVs) described above and below physically incorporate the polynucleotide to be delivered as a structural component of the SPDV. As such, the structure of the polynucleotide contributes to the structural characteristics of the SPDV. Typically, where the polynucleotide is in the form of a plasmid, the DNA will generally comprise either supercoiled or relaxed circles, or a mixture thereof. To the extent that a specific form may be preferred for a given application, enzymes such as DNA gyrase, ligase, and topoisomerase may be used to alter the structure of the plasmid as deemed necessary. Where linear polynucleotides are preferred, plasmids may be linearized, and optionally concatamerized, prior to complex formation.

Single- and double-stranded polynucleotides might also be “prepackaged” prior to lipid complex formation by the addition of suitable polynucleotide binding proteins such as viral proteins, single-stranded binding protein, histone proteins and the like.

Polynucleotides of interest which may be delivered using the claimed delivery vehicles include, but are not limited to, DNA, RNA, polynucleotides associated with procaryotic and eucaryotic viral particles (e.g., retroviral core particles, bacteriophage particles, adenovirus particles, adenoassociated virus core particles, and the like), protein/DNA complexes, i.e., proteins for integration, endosome disruption, to facilitate gene transfer and expression, etc.; RNA/DNA complexes, and any and all derivatives and variations of the above. Where a DNA molecule is to be delivered, it will typically comprise a gene of interest, or portion thereof, which is flanked by regulatory sequences which are spatially organized to optimize the expression of the DNA of interest.

Preferably, the polynucleotide to be delivered using the presently described SPDVs will be substantially pure (i.e., substantially free of contaminating proteins, lipid, polysaccharide, and nucleic acid). For example, where plasmid DNA is used, the preparations will generally be prepared by a process comprising phenol, or phenol:chloroform, extraction, and isopycnic centrifugation (using CsCl, and the like), or functional equivalents thereof. Preferably, the DNA preparations will also be treated with RNase, and subject to multiple rounds of extraction, and at least two rounds of ultracentrifugation. Typically, a substantially pure preparation of nucleic acid is a preparation in which at least about eighty percent, generally at least about ninety percent, and preferably at least about ninety five percent of the total nucleic acid is comprised of the desired nucleic acid.

In particular, genes of interest may be inserted into a wide range of expression vectors which may subsequently be delivered using the presently disclosed methods. Suitable vectors which may be delivered using the presently disclosed methods and compositions include, but are not limited to, herpes simplex virus vectors, adenovirus vectors, adeno-associated virus vectors, retroviral vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San Diego (1995). It is contemplated that the subject methods and compositions may be used to directly deliver vector nucleic acid, or, where applicable, viral or subviral particles encoding or containing the nucleic acid of interest.

As used herein, the term “expression” refers to the transcription of the DNA of interest, and the splicing, processing, stability, and, optionally, translation of the corresponding mRNA transcript. Depending on the structure of the DNA molecule delivered, expression may be transient or continuous.

Any number of transcriptional promoters and enhancers may be used in the DNA of interest, including, but not limited to, the herpes simplex thymidine kinase promoter, cytomegalovirus promoter/enhancer, SV40 promoters, and retroviral long terminal repeat (LTR) promoter/enhancers, and the like, as well as any permutations and variations thereof, which may be produced using well established molecular biology techniques (see generally, Sambrook et al. (1989) Molecular Cloning Vols. I-III, Cold Spring Harbor Laboratory Press, Cold Spring Harbor, New York, and Current Protocols in Molecular Biology (1989) John Wiley & Sons, all Vols. and periodic updates thereof, herein incorporated by reference). Promoter/enhancer regions may also be selected to provide tissue-specific expression.

DNAs of particular interest include, but are not limited to sequences encoding a variety of proteins, cytokines and growth factors, (such as, G-CSF, GM-CSF, nerve growth factor (NGF), ciliary neurotropic factor (CNTF), brain-derived neurotropic factor (BDNF), interleukins 1-2 and 4-14, tumor necrosis factor-α (TNF-α), α or γ interferons, erythropoietin, and the like), the cystic fibrosis transmembrane conductance regulator (CFTR), tyrosine hydroxylase (TH), D-amino acid decarboxylase, GTP cyclohydrolase, leptin, leptin receptor, factors VIII and IX, tissue plasminogen activator (tPA).

Additionally, antisense, antigene, or aptomeric oligonucleotides may be delivered using the presently described SPDVs. Ribozymes, RNA-DNA hybrids, polynucleotide peptide bonded oligos (PNAs), circular or linear RNA, circular single-stranded DNA.

RNAs of interest include self-replicating RNAs, mRNA transcripts corresponding to any of the above genes which may be directly translated in the cytoplasm, or catalytic RNAs, e.g. “hammerheads” hairpins, hepatitis delta virus, group I introns which may specifically target and/or cleave specific RNA sequences in vivo. Of particular interest for targeting by catalytic RNAs are RNA viruses, and both cell and viral transcripts.

Alternatively, antisense forms of RNA, DNA, or a mixture of both may be delivered to cells to inhibit the expression of a particular gene of interest in the cell or to correct point, or other (nonsense or missense, etc.) mutations.

An additional embodiment of the present invention contemplates the delivery of oligomeric nucleotides which have been incorporated into the SPDVs in conjunction with larger polynucleotides. Such “carrier” polynucleotides may be single-stranded (linear or circular), or substantially double-stranded, and may additionally comprise one or more regions which are substantially homologous or complementary to the oligomeric nucleotides to be delivered.

When desired, the DNA of interest may further incorporate a suicide signal that allows for the controlled extermination of cells harboring and expressing the DNA of interest previously delivered by the delivery vehicle. For instance, the thymidine kinase (tk) gene may be incorporated into the delivered DNA which would allow the practitioner to subsequently kill cells expressing the tk gene by administering the correct amounts of acyclovir, gangcyclovir, or the conceptual or functional equivalents thereof.

The claimed methods for producing stable polynucleotide delivery vehicles require that the polynucleotide(s) of interest be contacted with the amphipathic cationic lipid conjugates such that ion pairing between the cationic moiety and the polynucleotide allows the polynucleotide to condense. Typically, contacting is achieved by first dissolving the lipid constituent in a suitable detergent in order to form lipid micelles. Detergents suitable for dissolving lipids include, but are not limited to cholate, deoxycholate, lauroyl sarcosine, octanoyl sucrose, CHAPS (3-[(3-cholamidopropyl)-di-methylaminel-2-hydroxyl-1-propane), novel-β-D-glucopyranoside, Lauryl dimethylamine oxide, octylglucoside, and the like. Preferably, the detergent will be nonionic and possess a high critical micelle concentration (CMC). When the polynucleotide is added to the micellized amphipathic cationic lipid conjugate, ion pairing occurs and the polynucleotide condenses as a complex with the amphipathic cationic lipid conjugate.

Given that ion pairing plays a role in the formation of the stable lipid/polynucleotide complex, the pH during complex formation may be varied to optimize or stabilize the interaction of the specific components. For instance, where non-pH sensitive cationic lipids are used, a pH as low as about 4 may be preferred to complex a given polynucleotide (e.g., RNA) or other chemical agent which may be coincorporated with the polynucleotide. Additionally, where the polynucleotide (e.g., DNA) is not substantially sensitive to base hydrolysis, circumstances may dictate that a pH of up to about 10 be used during complex formation. Generally, a pH within the range of about 5 to about 9 will be used during complex formation and transfection.

Typically, many cationic condensing agents (e.g., spermine or spermidine) will precipitate polynucleotide. However, the carefully controlled addition of condensing cation to the polynucleotide (using an infusion pump or the like) allows for relatively high concentrations of polynucleotide (e.g., about 0.5 mg/ml) to be complexed with the condensing agent. As such, carefully controlled addition of the polynucleotide to the micellized cationic lipid condensing agent allows for relatively high concentrations of polynucleotide to be complexed by the cationic condensing agent.

After initial complex formation, slow removal of the detergent (i.e., by extensive dialysis) allows for the assembly and formation of stable polynucleotide delivery vehicles. While slow dialysis remains the preferred method of detergent removal, one may expedite detergent removal by increasing the relative amount of dialysis buffer or by adding a reagent to the buffer which binds and removes the detergent from the dialysace buffer solution.

Alternatively, the polynucleotide may be dissolved in a solution containing a suitable cation prior to the addition of cationic lipid and detergent. After the detergent is added, it is removed by dialysis in the presence of cation, and subsequently the cation may removed by dialysis. Suitable cations include any element carrying a positive charge. The cation may be monovalent, divalent, or multivalent. Typical examples of suitable cations include, but are not limited to manganese, magnesium, sodium, calcium, rubidium, zinc, molybdenum, nickel, iron and the like. Generally, the cation will be added in an amount sufficient to prevent aggregate formation during complexation of the lipid and the polynucleotide, and up to a concentration of about the maximum solubility of a given cationic compound. Preferably, the concentration of sodium, (e.g., sodium chloride) will be between about 0.1 molar and about 5 molar, the concentration of magnesium (e.g., magnesium chloride) will be between about 0.05 molar and about 5 molar; and the concentration of manganese (e.g., manganese chloride) will be between about 0.1 molar and about 4 molar.

Additionally, one of ordinary skill will appreciate that the type and concentration of cation may have to be adjusted depending on the type of detergent or cationic lipid used to assemble the SPDVs.

Where the polynucleotide, or oligonucleotide, is to be complexed with cation during the assembly of SPDVs, the cationic lipid and/or detergent may be added prior to, concurrently with, or subsequent to, the addition of cation. Generally, the cationic lipid will be added to the poly, or oligo nucleotide at a cationic lipid-to-polynucleotide phosphate ratio of between about 0.1:1 and about 16:1, preferably between about 0.5:1 and about 7:1, more preferably between about 0.7:1 and about 2:1, and specifically about 1:1. The above ratios are provided for exemplification and not limitation, and may be modified depending on the characteristics of the cationic lipid used to assemble the SPDVs. Also, the optional ratio will be dependent upon the DNA concentration.

After the cation, poly or oligonucleotide, cationic (or other) lipid, and detergent are present in the milieu, the detergent will preferably be removed by dialysis in cation comprising buffer After the detergent is removed, the cation may subsequently be substantially removed by dialysis, or a functional equivalent. Preferably, dialysis will be generally be performed at a temperature of between about 4° C. and about 30° C., and will result in a final cation concentration that is not detrimental to the intended use of the SPDV. For instance, the cation may be substantially removed by, for example, dialysis with a buffered solution that is suitable for parenteral administration.

After the substantial removal of the cation, the resulting SPDVs generally remain stable (i.e., retain transduction activity) for at least two weeks when stored at about 4° C.

Where the polynucleotide is complexed with cation (or otherwise precondensed) prior to or concurrent with the addition of cationic lipid, stable complexes may be formed (in the presence of detergent) at relatively low lipid:nucleic acid phosphate ratios. By using a lower ratio of lipid, a higher proportion of the lipid is incorporated in the complex which results in presence of less unincorporated lipid in the final product. This feature is desirable where a nonbiodegradable lipid is used because unincorporated forms of such lipids are substantially toxic to the target cells. Accordingly, another embodiment of present invention are SPDVs produced essentially as described above that comprise reduced toxicity. For the purposes of the present disclosure, reduced toxicity shall mean that SPDVs comprising at least about 10 μg of DNA may be injected into an animal without the animal suffered grave toxicity effects.

An additional feature of complexing or precondensing the nucleic acid with cation is that higher concentrations of DNA may be used to form the SPDVs. For example, the presence of MnCl₂ at 0.1 molar allows for SDPV formation at a concentration of about 0.05mg/ml of DNA. Similarly, by increasing the concentration of cation, one may increase the concentration of DNA used to assemble the SPDVs by a corresponding amount (i.e., 2 molar MnCl₂ may allow for SPDV formation at a concentration of about 1 mg/ml of DNA). Given the relatively high solubility of the applicable cations (i.e., NaCl saturates at about 5.5 molar), it is clear that the present methods enables the formation of SPDVs at a concentration of at least about 10 mg/ml of DNA (or other polynucleotide). Accordingly, another embodiment of the present invention are preparations of SPDVs that have been formulated as described above and comprise a concentration of DNA (or other nucleotide) of generally between about 0.05 mg/ml and about 10 mg/ml, preferably between about 0.25 mg/ml and about 10 mg/ml, more preferably between about 0.5 mg/ml and about 1.5 mg/ml, and specifically between about 0.8 mg/ml and 1.2 mg/ml. Accordingly, another embodiment of the present invention are SPDV compositions comprising high concentrations of nucleic acid (i.e. >0.25 mg/ml nucleic acid).

Because of the inherent stability of the polynucleotide delivery vehicles produced by the present methods, targeting agents may be stably incorporated into the vehicles to direct the vehicles to specific cells and/or tissues. Accordingly, prior to, or during detergent removal, any of a variety of targeting agents may be also be incorporated into the delivery vehicles.

For the purposes of this disclosure, the term targeting agent shall refer to any and all ligands or ligand receptors which may be incorporated into the delivery vehicles. Such ligands may include, but are not limited to, antibodies such as IgM, IgG, IgA, IgD, and the like, or any portions or subsets thereof, cell factors, cell surface receptors, MHC or HLA markers, viral envelope proteins, peptides or small organic ligands, derivatives thereof, and the like.

If necessary, the ligand may be derivatized to an appropriate lipid moiety prior to incorporation into the polynucleotide delivery vehicle. For example, the targeting agent (e.g., immunoglobulin) may be N-linked to a free carboxyl group of the polar region of a amphipathic lipid by first derivatizing a leaving group to the carboxyl group using N-hydroxysuccinimide (NHS) and 1-ethyl-3-(3-dimethylaminopropyl)carbodiimide (EDAC), or the methiodide thereof, (EDC methiodide) and a free amino group on the targeting molecule. Alternatively, targeting agents may be disulfide linked to a properly conditioned delivery vehicle/lipid (using thioacetic acid, hydroxylamine, and EDTA), or succinimidyl acetylthioacetate may be used in conjunction with a fatty acid (e.g., dioleylphosphatidyl-ethanolamine, DOPE) to form a DOPE-thioacetate (ATA) which may be treated with hydroxylamine to generate the reduced molecule (DOPE-acetyl-SH). A free amino group on the targeting agent is reacted with succinimidyl maleimidophenyl butyrate (SMPB) to produce a target which is linked to maleimidophenylbutyrate (MPB) by a peptide bond. The derivatized fatty acid is subsequently combined with the target-MPB complex to produce a targeting agent which has been cross-linked to a fatty acid.

Additionally, the targeting agent may be linked to the lipid by a biodegradable linkage as discussed above (peptide or dipeptide linkers, pH hydrolyzable linkers, etc.).

Alternatively, the targeting agent may also act as a bridge between the stable complex and the “targeted” cells or tissues. For instance, where the targeting agent simply associates with the complex, the agent may be added to the complex well after complex formation or isolation. To the extent that the targeting agent is also capable of recognizing, or being recognized, by molecules on the cell surface, it may act as a bridge molecule which effectively places the complex in intimate contact with the cell surface.

Particularly where hepatocytes are the preferred target of lipid-directed transfection, molecules such as fetuin may prove useful. Hepatocytes contain a galactose receptor. After treatment with neuraminidase, fetuin is converted to asialofetuin which displays a number of galactose residues on its surface. Moreover, both fetuin and asialofetuin are known to associate with the stable lipid/DNA complex.

As a molecule rich in acidic amino acids (aspartic acid and glutamate) asialofetuin (ASF) presumably associates the cationic head groups of the lipid complexes. Consequently, the asialofetuin-associated complexes are targeted to hepatocytes by virtue of the exposed galactose residues on the protein.

The observation that asialofetuin associates with the stable complexes also has far reaching potential. For instance, asialofetuin may be derivatized with any of a wide number to targeting ligands using any of a number of conventional chemical methods. For instance, periodate may be used to convert at least a portion of the hydroxyl groups on galactose to aldehydes, the aldehydes react with primary amino groups to form Schiff bases, which may be subsequently be reduced with lithium aluminum hydride (to add a targeting ligand). Alternatively, the aldehydes may be reacted with hydrazide to attach heterobifunctional cross-linking reagents (which has been to suitable targeting ligands). Either of the above strategies are simply illustrative of the many possible ways asialofetuin may be derivatized with practically any targeting ligand, and should not be construed as limiting the invention in any way.

After ligand association, the derivatized asialofetuin may be associated with the stable complex as described above. Virtually any ligand can be attached to asialofetuin, and virtually any DNA can be packaged into the stable complex. Thus, by carefully matching a properly derivatized asialofetuin with the appropriate stable complex, virtually any cell may be targeted to express virtually any gene.

Moreover, asialofetuin, or functional equivalents thereof (vis-a-vis binding) may be reacted with N-hydroxy-succinimidyl-oleate to produce an acylated, or multi-acylated asialofetuin derivative which may be directly incorporated into lipid complexes. Additionally, any of a wide variety of fatty acid chains may also be linked to ASF using this methodology. Typically, at least about 1 to about 20 acylated asialofetuin molecules will be incorporated per complex, and preferably about 5 to about 12 molecules will be incorporated.

Additionally, it is likely that other proteins will be identified or developed that are also capable of associating with stable cationic lipid/nucleic acid complexes. Like asialofetuin, proteins that associate with the stable complex may be suitably derivatized with a targeting ligand and used to direct the stable complex to specific cells and tissues. In this manner, any of a variety of cells such as endothelial cells, line cells, epithelial cells, islets, neurons or neural tissue, mesothelial cells, osteocytes, chondrocytes, hematopoietic cells, immune cells, cells of the major glands or organs (e.g., lung, heart, stomach, pancreas, kidney, skin, etc.), exocrine and/or endocrine cells,- and the like.

Of particular interest are the proteins encoding various cell surface markers and receptors. A brief list that is exemplary of such proteins includes: CD1(a-c), CD4, CD8-11(a-c), CD15, CDw17, CD18, CD21-25, CD27, CD30-45(R(O, A, and B)), CD46-48, CDw49(b,d,f), CDw50, CD51, CD53-54, CDw60, CD61-64, CDw65, CD66-69, CDw70, CD71, CD73-74, CDw75, CD76-77, LAMP-1 and LAMP-2, and the T-cell receptor, integrin receptors, endoglin for proliferative endothelium, or antibodies against the same.

For the purposes of the present disclosure, “stable” polynucleotide delivery vehicles generally retain transfection efficiencies of at least about twenty (20) percent of the polynucleotide transfection efficiency of freshly prepared product after storage for forty-eight (48) hours, and preferably retain at least about thirty-five (35) percent transfection efficiency after 48 hours, and in a particularly preferred embodiment will retain at least about fifty (50) percent transfection efficiency after 48 hours.

Alternatively, the presently described stable polynucleotide delivery vehicles remain size-stable and generally retain a discrete size range of between about 30 and about 200 nm, preferably between about 50 and about 150 nm, and preferably between about 70 and about 115 nm average particle size (as per a Gaussian distribution) after being held in the liquid state for at least 48 hours.

Where stability in serum is concerned, the present stable polynucleotide delivery vehicles are serum stable in that they are generally at least about twice as stable than, and preferably at least about one order of magnitude more stable than liposomal formulations produced using the methods/synthetic cationic lipids taught by the prior art when exposed to serum concentrations of up to about fifteen (15) percent.

The stability of the presently described SPDVs may be augmented by the appropriate storage conditions. For example, the SPDVs may be frozen and stored indefinitely. After rapid or slow (at about 4° C.) thawing, the SPDVs typically retain a substantial portion, if not all, of the transfection efficiency of freshly produced samples. Moreover, the subject SPDVs also retain a substantial amount (i.e., at least about 50 percent) of their original transfection efficiency after lyophilization and reconstitution.

Where one seeks to augment long-term stability by freezing or freeze-drying the SPDVs, suitable excipients may be added to the SPDV preparation prior to freezing. Examples of such stabilizing excipients include, mono or disaccharides (e.g., glucose, sucrose, etc.), polysaccharides, or any of a variety of well-known agents (e.g., glycerols, gums, dextrans, and the like).

Stable lipid/DNA complexes may aggregate. In particular, stable complexes formed at lipid to DNA ratios of greater than about 0.5, generally above about 1.0, and preferably at least about 3.0 up to about 25 may form loose aggregates when held at temperatures between room temperature to about 0° C., generally between about 15° C. and about 1° C., and preferably about 4° C. For the purposes of this disclosure, a loose aggregate is defined as an aggregate that is easily dispersible into suspension. Optionally, such aggregation may occur after a period of frozen storage (at about −20° C. or less), followed by thawing. To the extent that aggregation is desirable, the level of aggregation may be regulated by any of a number of means in addition to adjusting temperature. For example, buffer/salt concentration may be adjusted to increase the amount of aggregation. Moreover, coprecipitants may be added which complex with the stable complexes and further increase the rate of extent of precipitation. Aggregation may also be increased by the addition of facilitating agents. For example, where a targeting agent or receptor is incorporated into the complex, a suitable lectin, ligand, or antibody may be added to cross-link the complexes and increase the rate and extent of aggregation or precipitation.

Optionally, a suitable ligand or antibody, or mixture thereof, may be affixed to a suitable solid support, i.e., latex beads, microcarrier beads, membranes or filters, and the like, and used to selectively bind complexes which incorporate the targeting receptor or ligand from solution. Thus, stable complexes may also be selectively removed from solution using this method.

Alternatively, one may substantially detoxify complexes made with nonbiodegradable cationic lipids by taking steps to remove the excess unassociated lipid prior to dialysis. For example, noncomplexed lipid will generally be present as a lipid micelle in detergent solution. As such, size exclusion chromatography, in the presence of detergent, may be used to separate the noncomplexed lipid (i.e., toxic component) from the larger stable complex (which will presumably elute in the void volume).

The aggregated stable lipid/DNA complexes will optimally retain transfection activity. Moreover, the aggregated complexes may be concentrated by a variety of standard techniques including, but not limited to, centrifugation (where they effectively form a precipitate) and/or vacuum or simple evaporation, ultrafiltration, chromatofocusing, electrophoresis, column chromatography, and the like. After isolation, the concentration of the aggregated complexes may be adjusted by gently resuspending or diluting the complexes with a suitable buffer. Where in vivo use of the concentrated stable complexes is contemplated, the buffer will be suitable for in vivo administration.

As a net result of isolation and resuspension, stable complexes may be obtained which both retain DNA transfection activity, and comprise DNA concentrations which far exceed the amounts of DNA that may normally be loaded into conventionally produced lipid/DNA complexes. Presently, transient and stable complexes produced at similarly high DNA/lipid concentrations lack measurable transfection activity (due to high toxicity).

The net effect of this limitation is that the relatively small amount of nucleic acid that may typically be introduced by conventionally produced lipid-based transfection methods has generally blocked more widespread use and application of the technology. Thus, the presently demonstrated ability to concentrate the stable complexes represents yet another novel aspect of the present invention. Accordingly, an additional embodiment of the present invention is a method of producing stable lipid/nucleic acid complexes that retain measurable transfection activity, and comprise at least about 10 μg of nucleic acid per ml up to about 10 mg/ml.

In addition to enabling the use and delivery of high concentrations of genetic material, aggregation also results in a drastic reduction of the toxicity which is an inherent feature of traditional lipid-mediated methods for the delivery of nucleic acid to cells. The nonbiodegradable cationic lipids typically used in lipofection are relatively toxic to cells. Thus, target cells can only tolerate limited concentrations of cationic lipid. During cationic lipid/DNA complex formation, a significant portion of the input cationic lipid does not associate into the DNA complex (i.e., does not facilitate DNA transfection). Given that complex formation involves relatively fixed ratios of lipid/DNA, it necessarily follows that correspondingly fixed percentage of unincorporated lipid will be present in any sample of lipid/DNA complex. Since increasing the amount of DNA offered to cells also increases the net amount of (toxic) lipid, the presence of the unincorporated lipid severely limits the amount of genetic material that may be offered to target cells. Thus, the act of separating the transfection activity (i.e., lipid/DNA complex) from the free unassociated lipid, essentially increases the net amount of nucleic acid that may be offered to the target cells.

Toxicity studies using the aggregated and resuspended stable complexes indicate that the vast majority of the toxic activity remains in the supernatant. Conversely, the resuspended aggregates retain high levels of transfection activity while exhibiting far less toxicity than transient complexes formed at corresponding lipid/DNA ratios. Thus, by removing the stable complexes from suspension, one is able to effectively isolate the transfection activity away from the source of the major source of cellular toxicity. Accordingly, another embodiment of the present invention is an isolated stable cationic lipid/nucleic acid complex which has substantially reduced toxicity relative to transient or stable complexes that have been formed at similar lipid/nucleic acid ratios and/or lipid/nucleic acid concentrations.

Similarly, another embodiment of the present invention is a method of producing stable cationic lipid/nucleic acid complexes of substantially reduced toxicity. For the purposes of this disclosure, the term “substantially reduced toxicity” shall mean that the toxicity of an agent shall generally be reduced by at least about 25 percent relative to untreated agent, preferably by at least about 50 percent, and optimally a reduction of at least about a 100 percent will be achieved. Toxicity may also be measured by determining the dose which is lethal to fifty percent of the test subjects. Generally, the described stable polynucleotide vehicles of substantially reduced toxicity will have a lethal dose, or LD₅₀, twice that of nonisolated stable complex formed at similar cationic lipid/phosphate ratios, and optimally reduced toxicity vehicles will have an LD₅₀ at least about one order of magnitude greater than that of corresponding nonisolated cationic lipid/polynucleotide mixtures.

Given the novel methods of production described above, yet another embodiment of the present invention describes stable polynucleotide delivery vehicles comprising amphipathic cationic lipid conjugates and/or the above-described biodegradable cationic lipid conjugates. When appropriate, any or a variety (i.e., mixture) of other “helper” lipid moieties may also be added to the stable polynucleotide delivery vehicles to alter the chemical characteristics of the vehicle. As such, any of a number of well known phospholipids may be added including, but not limited to, disteroylphosphatidyl-glycerol (DSPG), hydrogenated soy, phosphatidyl choline, phosphatidylglycerol, phosphatidic acid, phosphatidylserine, phosphatidylinositol, phosphatidyl ethanolamine, sphingomyelin, mono-, di-, and triacylglycerols, ceramides, cerebrosides, phosphatidyl glycerol (HSPG), dioleoyl-phosphatidylcholine (DOPC), cardiolipin, and the like. Additional lipid moieties include cholesterol, cholesterol esters, and other derivatives thereof. Where appropriate, it may be preferable that the additional lipids and phospholipids be positively or negatively charged, and the ratios of various lipid moieties may be altered to effect the optimum charge ratios.

Additionally, any of a variety of stabilizing agents may be utilized in conjunction with the described vehicles. Although oxidation of the various components may be substantially reduced by preparing formulations in accordance with the present invention under an inert atmosphere, such as nitrogen, this is a somewhat inconvenient and expensive process and so it is often preferred to add chemical anti-oxidants. Suitable pharmaceutically acceptable antioxidants include propyl gallate, butylated hydroxyanisole, butylated hydroxytoluene, ascorbic acid or sodium ascorbate, DL- or D-alpha tocopherol and DL- or D- alpha-tocopheryl acetate. The anti-oxidant, if present, may be added singly or in combination to the polynucleotide delivery vehicles either before, during, or after vehicle assembly in an amount of up to, for example, 0.1% (w/v), preferably from 0.0001 to 0.05%.

One of ordinary skill will appreciate that, from a medical practitioner's or patient's perspective, virtually any alleviation or prevention of an undesirable symptom (e.g., symptoms related to disease, sensitivity to environmental or factors, normal aging, and the like) would be desirable. Thus, for the purposes of this Application, the terms “treatment”, “therapeutic use”, or “medicinal use” used herein shall refer to any and all uses of the claimed compositions which remedy a disease state or symptoms, or otherwise prevent, hinder, retard, or reverse the progression of disease or other undesirable symptoms in any way whatsoever.

When used in the therapeutic treatment of disease, an appropriate dosage of stable polynucleotide delivery vehicle (SPDV), or a derivative thereof, may be determined by any of several well established methodologies. For instance, animal studies are commonly used to determine the maximal tolerable dose, or MTD, of bioactive agent per kilogram weight. In general, at least one of the animal species tested is mammalian. Those skilled in the art regularly extrapolate doses for efficacy and avoiding toxicity to other species, including human. Before human studies of efficacy are undertaken, Phase I clinical studies in normal subjects help establish safe doses.

Particularly where in vivo use is contemplated, the various biochemical components of the present invention are preferably of high purity and are substantially free of potentially harmful contaminants (e.g., at least National Food (NF) grade, generally at least analytical grade, and preferably at least pharmaceutical grade). To the extent that a given compound must be synthesized prior to use, such synthesis or subsequent purification shall preferably result in a product that is substantially free of any potentially toxic agents which may have been used during the synthesis or purification procedures.

Additionally, stable polynucleotide delivery vehicles (SPDVs) may also be modified to enhance in vivo stability as well as any of a variety of pharmacological properties (e.g., increase in vivo half-life, reduce toxicity [although no in vivo toxicity has been observed for DMRIE/DOPE (1:1, mol:mol), DOSPA/DOPE (4:1), or PC-cholesterol (chol)/DOPE (1:1)], etc.) by established methods. For instance, using simple long circulating lipid compositions such as disteroylphosphatidylcholine (DSPC)/chol (2:1), or sphingomyelin/chol (1:1) may be used to test half-life/toxicity. Alternatively, inclusion of synthetic polyethylene PC-chol/DOPE (1:1) phospholipids or gangliosides, such as GM₁, may be used to increase circulation half-lives. Toxicity may be further reduced by using cationic lipids with biodegradable linkages such as esters or ceramides which join the acyl chains to the cationic moiety, or carbamate, hydrazide, or anhydrides linkages to join the acyl chains to the cationic groups.

Where diagnostic, therapeutic or medicinal use of SPDVs, or derivatives thereof, is contemplated, the SPDV may prepared and maintained under sterile conditions and thus avoid microbial contamination. Because of the relatively small size and inherent stability of the SPDVs, compositions comprising SPDVs may also be sterile filtered prior to use. In addition to the above methods of sterile preparation and filter sterilization, antimicrobial agents may also be added. Antimicrobial agents which may be used, generally in amounts of up to about 3% w/v, preferably from about 0.5 to 2.5%, of the total formulation, include, but are not limited to, methylparaben, ethylparaben, propylparaben, butylparaben, phenol, dehydroacetic acid, phenylethyl alcohol, sodium benzoate, sorbic acid, thymol, thimerosal, sodium dehydroacetate, benzyl alcohol, cresol, p-chloro-m-cresol, chlorobutanol, phenylmercuric acetate, phenylmercuric borate, phenylmercuric nitrate and benzylalkonium chloride. Preferably, anti-microbial additives will either enhance the biochemical properties of the SPDVs, or will be inert with respect SPDV activity. To the extent that a given anti-microbial agent may prove deleterious to SPDV activity, another agent may be substituted which effects SPDV function to a lesser extent.

Compositions comprising SPDVs as active components may be introduced in vivo by any of a number of established methods. For instance, the agent may be administered by inhalation; by subcutaneous (sub-q); intravenous (I.V.), intraperitoneal (I.P.), or intramuscular (I.M.) injection; rectally, as a topically applied agent (transdermal patch, ointments, creams, salves, eye drops, and the like), or directly injected into tissue such as tumors or other organs, or in or around the viscera.

Another embodiment of the subject invention involves the use of SPDVs to effect gene therapy. Such gene therapy is intended to compensate for genetic deficiencies in the afflicted individual's genome and may be effected by ex vivo isomatic cell gene therapy whereby host cells are removed from the body are transduced to express the deficient gene and reimplanted into the host. Alternatively, somatic cell gene therapy may be effected by directly injecting a vector bearing the desired gene into the individual, in vivo, whereby the gene will be delivered and expressed by host tissue.

Although the above polynucleotide delivery vehicles are primarily intended to provide polynucleotides to cells, a further embodiment of the present invention contemplates the packaging and delivery of any of a variety of suitable bioactive agents in addition to polynucleotides. For instance, to the extent that a bioactive agent (e.g., any protein, peptide, small organic molecule, and the like) of interest comprises a net negative charge or comprises a substantial amount of negatively charged character, it may prove useful to deliver such an agent using biodegradable amphipathic lipids incorporated into established liposomal formulations or lipid emulsions, or deliver vehicles constructed by the methods substantially analogous to those presently disclosed.

In addition, to the extent that a given agent of interest may associate with polynucleotide (e.g., proteins or other molecules with DNA and/or RNA binding activity), it may prove useful to deliver the agents to the body by first incorporating them into polynucleotide delivery vehicles.

Additionally, given that liposomes and lipid emulsions or microemulsions have proved useful as drug delivery tools or as components of cosmetics, it is contemplated that the presently disclosed novel biodegradable amphipathic lipids may also prove useful as stabilizing, structural, or binding components of lipidic structures, membranes, or emulsions. Typically, the biodegradable amphipathic lipids will be either added or interchanged with lipid components of existing formulations, or novel formulations may be constructed which take further advantage of the charged group of the amphipathic lipid.

If desired, one or more stabilizers and/or plasticizers may be added to the emulsions and liposomal formulations for greater storage stability. Although microemulsions tend not separate upon standing under normal conditions, a greater degree of stability may be useful under some circumstances. Materials useful as stabilizer and/or plasticizer include simple carbohydrates including, but not limited to, glucose, galactose, sucrose, or lactose, dextrin, acacia, carboxypolymethylene and colloidal aluminum hydroxide. When stabilizers/plasticizers are added, they may be incorporated in amounts up to about 10% (w/v), preferably from about 0.5 to 6.5%, of the total preparation.

Lipid formulations (e.g., emulsions, microemulsions, liposomes, or delivery vehicles) comprising the disclosed biodegradable amphipathic cationic lipids may also significantly protect the encapsulated bioactive agents from the digestive process. As such, the formulations may also prove useful for the oral administration of bioactive agents.

To the extent that additional enteric protection is desired, for added protection, it is possible to formulate solid or liquid formulations in accordance with the invention in an enteric-coated or otherwise protected form. In the case of liquid formulations, they can either be mixed or simply coadministered with a protectant, such as a liquid mixture of medium chain triglycerides, or they can be filled into enteric capsules (for example of soft or hard gelatin, which are themselves optionally additionally enteric coated. Alternatively, solid formulations may be treated more flexibly. They may either be coated with enteric materials to form tablets or they can be filled into enteric capsules.

The thickness of enteric coating on tablets or capsules can be, for example, from 0.5 to 4 microns in thickness, although the precise thickness will be determined by the skilled formulator. Enteric coated granules (whose particle size may be, for example, from 0.5 to 2 mm) may themselves be coated without being compounded into a tablet for coating. Microcapsules, similarly, can be enteric coated. The enteric coating may comprise any of the enteric materials conventionally utilized in orally administrable pharmaceutical formulations. Suitable enteric coating materials are known, for example, from “Remington's Pharmaceutical Sciences”, 15th Edition, pp. 1614-1615 (1975); 2nd Edition, pp. 116-117, 371-374 (1976); and “Hagars Handbuch der Pharmazeutischen Praxie”, 4th Edition, Volume 7a (Springer Verlag 1971), pages 739 to 742 and 776 to 778.

Examples of suitable enteric coating materials include cellulose acetylphthalate, hydroxypropylmethylcellulose-phthalate (HPMC-P), benzophenyl salicylate, cellulose acetosuccinate, copolymers of styrene and maleic acid, formulated gelatin, keratin, stearic acid, myristic acid, polyethylene glycol, shellac, gluten, acrylic and methacrylic resins and copolymers of maleic acid and phthalic acid derivatives. The enteric coating material(s) may be dissolved in solvents such as dichloromethane, ethanol and water, cellulose phthalate, or polyvinyl acetate phthalate. It is preferred to utilize HPMC-P, polyethylene glycol 6000 or shellac as the enteric coating. A proprietary preparation of HPMC-P aimed at dissolution or dissipation at pH 5.5, which is encountered in the human pyrolus, is available under the trade mark HP5-5, and is particularly preferred.

The term “biologically active material” includes, in particular, pharmaceutically active proteinaceous materials, and pharmaceutically active organic molecules. The proteinaceous material may be a pure protein, or it may comprise protein, in the way that a glycoprotein comprises both protein and sugar residues. The material may be useful in human or veterinary medicine, either by way of treatment or prophylaxis of diseases or their symptoms, or may be useful cosmetically or diagnostically. Examples of proteinaceous biological material which may be used in accordance with this invention include, but are not limited to, protein hormones such as insulin, calcitonin and growth hormone, whether from human or animals or semi- or totally synthetically prepared, erythropoietin, plasminogen activators and their precursors, such as tPA, urokinase, pro-urokinase and streptokinase, interferons including human interferon alpha, interleukins including IL-1, IL-2, IL-3, IL-4, IL-S, IL-6, IL-7 and IL-12, and blood factors including Factor VIII.

In any event, where they are biodegradable, the presently disclosed biodegradable amphipathic cationic lipids, and polynucleotide delivery vehicles produced therewith, represent a marked improvement over currently available synthetic cationic lipids vis-a-vis polynucleotide delivery to cells because the byproducts of the degradation reaction are substantially nontoxic, or inherently biocompatible. As such, the presently disclosed biodegradable amphipathic cationic lipids are be useful for the delivery of polynucleotides to cells in vitro as well as in vivo.

Another embodiment of the present invention is the use of the biocompatible pH sensitive or otherwise biodegradable linker portion of the amphipathic cationic lipid molecule to attach other biocompatible or groups to lipid moieties in place of the presently disclosed cationic groups. For instance, the above-mentioned targeting or bioactive proteins may be functionally derivatized to lipids and released into the cell after endocytosis. Alternatively, biocompatible anionic groups may also be attached to lipids in order to facilitate the complexation or encapsulation of positively charged bioactive agents or polymers into lipidic structures.

The examples below are provided to illustrate the subject invention. Given the level of skill in the art, one may be expected to modify any of the above or following disclosure to produce insubstantial differences from the specifically described features of the present invention. As such, the following examples are provided by way of illustration and are not included for the purpose of limiting the invention.

### 6.0 EXAMPLES

6.1 Methods of Making Biodegradable Cationic Lipid Conjugates

The biodegradable cationic lipids shown in FIG. 1(a) were synthesized as follows: 10 umol of N-glutaryl-DOPE (Avanti Polar Lipids, AL) in chloroform was added to a test tube. The chloroform was removed by evaporation under nitrogen to yield a lipid film. The residual chloroform was removed by vacuum. The film was hydrated in water and sonicated to form liposomes approximately 80 nms in diameter. A 5 fold molar excess of N-hydroxysuccinimide in 50 mM MES pH 6.2 and a 4 molar excess of EDC in 50 mM MES, pH 6.2 was added to the N-G-DOPE and incubated at room temperature for 20 minutes. Extraction of the lipid mixture was followed by thin layer chromatography analysis using chloroform/methanol/water (65/25/4) which showed that all the N-G-DOPE was converted to N-hydroxysuccinimide-N-G-DOPE under the conditions. The unreacted EDC, NHS, and the MES was removed by applying the reaction mixture to a three ml spin column containing Sephadex G-25 superfine equilibrated with 10 mM Hepes, pH 8.5. The liposomes were dissolved in 1 percent octylglucoside and the polyamine was added to the lipid in up to 5 fold molar excess to consume all of the NHS-N-G-DOPE. The reaction proceeded overnight at 4° C. The lipid product was dialyzed against water to remove the detergent, buffer and unreacted polyamine. The lipid product can then be solubilized in 1 percent octylglucoside and added to DNA using the essentially the same protocol as used for Lipofectamine/DNA complex formation, infra.

The biodegradable lipids shown in FIG. 1(b) (pentaethylenehexamine (PEHA) linked to N-succinyl-DOPE (NS-DOPE) or N-glutaryl-DOPE NG-DOPE) by an amide linkage, or PEHA linked to DOSG by an amide linkage) were prepared by reacting PEHA with either N-hydroxysuccinimidyl esters of NS-DOPE, NG-DOPE, or DOSG (prepared by treatment with EDC) at room temperature for 120 min. at pH 6.5 to about 8.5.

Alternatively, spermine may be linked to DOPE by a heterobifunctional crosslinking agent as follows. DOPE is derivatized with SMBP to yield a terminal maleimide on the head group (DOPE-MPB). Spermine is reacted with succinimidyl-acetyl-thioacetate to yield a spermine with a protected thiol. The protecting group is subsequently removed by hydroxylamine and the DOPE MOB is reacted with the thiol group on the spermine to yield DOPE BPM S-spermine. Using this scheme, the DOPE group remains susceptible to phospholipase hydrolysis thereby releasing the lipid from the DNA.

Another approach involves reacting SPDP (succinimidyl-pyridylyldithiopropionate) with DOPE to yield DOPE-PDP. After DOPE-PDP is reduced with DTT an exposed thiol group results. The thiol group on the DOPE may subsequently be reacted with the free thiol group of the modified spermine molecule described above to produce DOPE-S-S-spermine (DOPE linked to spermine by a disulfide linkage). The disulfide linkage is susceptible to reduction by the target cell which effectively results in the release of the DNA from the lipid.

FIG. 2 shows three strategies for synthesizing pH labile cationic lipids. In regard to Synthesis 1, reference is made to Blattler, W. A., et al., New heterobifunctional protein cross-linking reagent that forms an acid labile link Biochemistry 1985, 24, 1517-1524, and in regard to Synthesis 3, reference is made to Behr, J. P., et al., Efficient gene transfer into mammalian primary endocrine cells with lipopolyamine-coated DNA Proceedings from the National Academy of Sciences (USA), 1989, 86, 6982-6986. The disclosures of which are incorporated herein by reference.

Additionally, DOPE can be reacted with iminothiolane to yield a thiolated lipid. Apolyamine, such as spermine, can be reacted with iminothiolane as well. Addition of the derivatized spermine and lipid together in the presence of iodine will yield a polyamine-lipid linked by a disulfide.

6.2 Methods of Making Stable Delivery Vehicles

6.2.1 Reagents

Lipofectamine was purchased from Life Technology Inc, Gaithersburg, MD. Octylglucoside was purchased from SIGMA Chemical Co., St. Louis, Mo. A 7.3 kilobase, double stranded, closed circle DNA (pCMVβ, purchased from Clontech, Palo Alto, Calif.) containing a beta-galactosidase gene was grown in bacteria and isolated using a Quiagen MaxiPrep Isolation kit. (LPS)was removed by adsorption using a polymyxin B agarose column purchased from BioRad, Sunnyvale, Calif.

6.2.2 Protocol for the formulation of stable DNA/Lipid complexes

The following amounts of Lipofectamine (DOSPA), were solubilized in 1% octylglucoside, 10 mM TRIS, pH 7.4 and added to 5 ug of DNA (pCMVβ) in a total volume of 0.5 ml: 4.5 μl, 9 μl, 22.5 μl, 45 μl and 90 μl. Samples were dialyzed against 5% glucose, 10 mM TRIS, pH 7.5, at 4° C. Dialysis buffer was changed 5 times over the course of 48 hours. The resulting polynucleotide delivery vehicles were tested for particle size, and transfection efficiency as described below.

Similar methods were used to produce stable complexes with the cationic lipid DOTAP, and DC-CHOL (3-beta-(n-(N′,N′-dimethylaminoethane)carbamoyl)cholesterol).

6.3 Particle Size Analysis

Particle size analysis was obtained using a Leeds and Northrop laser dynamic light scattering instrument. Characterization of the transient (e.g., prior art) lipid/DNA complex with Lipofectamine showed that the starting size of the liposomes is approximately 50 nm in diameter. Addition of DNA caused the size to increase to 100 nm. As seen in FIG. 4, storage of these complexes over time showed a decrease in the 100 nm particles and an increase in much larger particles (d>1 μm). Storage of the transient lipid/DNA complex resulted in a decrease in gene expression.

Conversely, particle size analysis of the presently disclosed stable lipid/DNA complexes showed a very tight population of particles of about 100 nm. This same size particle was predominantly observed 48 hours after production. Fourteen days later, particle analysis showed a small percentage of larger particles (about 1 micron) with the predominant particle size being the 100 nm particles (see FIG. 5).

The fact that the rate of increase in particle size correlated with the rate of decrease in gene expression strongly suggests a direct relation between the two parameters.

6.4 Cell Transfection with Stable DNA/Lipid Complexes

Lipofectamine contains a cationic lipid abbreviated DOSPA. This lipid is composed of a spermine derivatized with a dialkyl ether. Transfection was examined in terms of the ratio of DOSPA to DNA phosphate. The stable lipid complexes were added to serum free media, DMEM and added to NIH-3T3 cells. Cells were incubated with the lipid/DNA complexes for 3 hrs. The media was removed and replaced with complete media. Incubation was continued for 48 hours. Cells were harvested in 0.1 ml of lysis buffer (0.1% TX-100,), and 0.040 ml of cells lysate was mixed with 0.05 ml of 2× concentration of beta-galactosidase substrate to measure enzyme activity. The level of beta-galactosidase expression was measured for both stable and transient complexes. The following experiments were performed:

6.4.1 Gene Expression

DOSPA is composed of spermine derivatized with a diacylether. While determining transfection efficiency, the spermine to DNA phosphate ratio was varied from between 0.5:1 and 10:1 (mol/mol). DNA transfection of the stable complex was compared to the transient complex which consisted of adding lipid and DNA together just prior to addition to the cells. NIH-3T3 cells were incubated with the cells for 3 to 4 hours at 37° C. in serum free media. The media was changed with complete media and the incubation was continued for 24 hours. Cells were harvested and lysates were analyzed for beta-galactosidase activity. The results are shown in FIG. 6 which shows that the stable complexes (solid line, left panel) gave the same level of gene expression as the transient complex (solid line, right panel). Secondly the optimal spermine to DNA phosphate ratio was the same for both complexes (about 2.5). Toxicity was observed for both the stable and transient complex at the higher spermine to DNA phosphate ratios (5-10:1). For this reason the DNA dose was reduced to 0.2 ug per well and the same ratios were tested. This resulted in a decrease in the amount of lipid added to the cells.

A reduction in the amount of DNA offered increased the optimal spermine to DNA phosphate ratio from 2.5 to 10 for both formulations, and the level toxicity observed (as determined by cell morphology) was substantially reduced. The striking difference is that the level of gene expression did not change for the stable complex (FIG. 6, dotted line, left panel) whereas expression by transient complex decreased by five-fold (FIG. 6, dotted line, right panel). The above results were expanded upon by keeping the spermine to DNA phosphate ratio at 10 and varying the amount of DNA used in complex formation. The results are shown in FIG. 7.

In FIG. 7, beta-galactosidase activity is graphed on a log scale on the Y axis and the DNA input dose is on the X axis. At the lowest dose of DNA used, only the stable complex showed gene expression. The subsequent doses showed that the stable complex yielded 5 to 10 fold higher levels of gene expression (and hence transfection efficiency) compared to the transient complex.

6.4.2 Stability Studies

The advantage of the stable complex over the transient complex was further illustrated in a comparison study where the efficiency of gene transfection was correlated with storage time at 4° C. The complexes were prepared at the same time and tested at the times indicated by the X axis for transfection of NIH-3T3 cells using the same cell transfection protocol as described above. The results are shown in FIG. 8(a). The transient complex displayed a dramatic drop in beta-galactosidase activity after 24 hours, and after 48 hours no expression was observed. Conversely, the stable complex displayed levels of activity after 48 hours which were equivalent to the levels of activity observed using freshly produced transient complex (forming the stable complex required 48 hours of dialysis, therefore the first time point is at 48 hours), and still retained about 50% of maximal transfection efficiency after 14 days of storage. These data were gathered without efforts to optimize storage conditions that would minimize loss of DNA transfection efficiency. Hence, the results represent the minimum level of stability enhancement which may be obtained using SPDVs as compared to the transient complex.

Long term stability studies were conducted using stable complex comprised of a DOSPA/DNA ratio of 6.6. Aliquots of the stable complex were stored at 20° C., −20° C. in the presence or absence of 5% dextrose, 4° C., room temperature, and 37° C. for up to ninety days. Stability was evaluated by assaying the transfection efficiency of the complexes. The results are shown in FIG. 8(b). FIG. 8(b) shows that stable complexes stored at 4° C. and −20° C. with 5% dextrose both retained substantially one hundred percent of initial transfection activity for at least 90 days, or at least 270 days when stored frozen. Stable complex stored at −20° C. without dextrose showed an initial five-fold decrease in transfection activity with a continued loss in activity over time, and stable complex stored at 37° C. lost essentially all transfection activity after 14 days. Conversely, the transient complex lost essentially all activity after as little as twenty-four hours. Lyophilization of the 5% dextrose storage condition yielded complete retention of activity when resuspended in volumes of 0.1x-1x of the original volume (i.e., could be concentrated by at least ten fold)

6.4.3 Demonstration of Serum Stability

An additional problem associated with transient cationic lipid/DNA complexes is inactivation by serum components. To avoid serum inactivation, current methods of transfection require that the cationic lipid/DNA complexes be incubated with the cells in serum-free media. This is a problem that largely forecloses the use of transient cationic lipid/DNA complexes in vivo. For this reason, serum stability with respect to transfection was tested. The serum was tested from 0 to 15% for both the stable complex and the transient complex. For these studies, 0.2 μg of DNA was used and the spermine to DNA phosphate ratio was held at 10:1. The results are shown in FIG. 9. The transient complex showed a marked drop in activity at 2 percent serum whereas the transfection efficiency of the stable complex decreased to a much lesser extent at all serum concentrations used. Subsequent X-gal staining studies have shown that about 10 percent of the exposed cells express β-gal at the tested range of serum concentrations.

The above data demonstrate: 1) The formation of a DNA/cationic lipid complex that remains stable for at least 14 days; 2) The transfection efficiency of the stable complex is greater than that of the presently known transient complex (as measured by gene expression); and 3) The stable complex is less susceptible to serum component inactivation than the transient complex.

6.5 Isolation and concentration of stable cationic lipid/DNA complexes.

Stable polynucleotide delivery vehicles were formed as described above using lipid to DNA phosphate ratios of 3.3, 6.6, and 16.5. The complexes comprised DOSPA and a 7.3 kb plasmid encoding, inter alia, the beta-galactosidase gene under the transcriptional control of a cytomegalovirus (CMV) promoter (pCMVβ).

The vehicles/complexes were stored at −20° C. in the presence of dextrose (five percent), and 4° C. After 90 days of storage, all of the samples retained essentially all of their initial transfection efficiency. During storage, the samples held at 4° C. accumulated a precipitate. The precipitate was separated from the solution by centrifugation at 3,000 g, for 15 min at 4° C., and the pellet was resuspended in 10 mM Tris. Both the resuspended precipitate, the supernatant, and the starting mixture were subsequently tested for transfection activity in 3T3 cells.

The results of the transfection experiments are shown in FIG. 10. FIG. 10 shows that the majority of the transfection activity remained in the supernatant when complexes were formed at a DOSPA/DNA ratio of 3.3; and that about 90 percent of the transfection activity apportioned to the pellet in the samples made at DOSPA/DNA ratios of 6.6 and 16.5. Lipid analysis of the 6.6 and 16.5 ratios showed little difference in the DOSPA/DOPE ratio, and indicated that approximately 10 percent of the total lipid remained in the pellet whereas 90 percent of the input lipid remained in the supernatant. Additionally, when the supernatant and concentrated complexes were further analyzed using thin layer chromatography (TLC), both the supernatant and the pellet (complex) contained identical ratios of DOSPA to DOPE. These data further verified that the majority of the lipid remained in the supernatant.

When the pellets resulting from preparations with lipid/DNA phosphate ratios of higher that about 6 were resuspended in 1/50th of the original volume, the concentrated compositions displayed transfection efficiencies which were substantially higher (up to several fold) than corresponding unconcentrated preparations. Presumably, the reason for the higher levels of transfection is due to the removal of uncomplexed lipid. As seen in FIG. 10, the uncomplexed lipid is largely responsible for the observed toxicity that is often associated with nonbiodegradable cationic lipids. Accordingly, by isolating the active component from the toxic component, one may add more DNA to the target cells which consequently increases the amount of gene transfer.

The significance of this observation is that unconcentrated complexes which are made using DNA concentrations approaching those contained in the concentrated sample display practically no transfection activity. Since the optimum ratios of lipid to DNA are relatively fixed, increasing the concentration of DNA requires that the concentration of lipid be increased by a proportionate amount. However, given that nonbiodegradable cationic lipids are often highly toxic to cells, only a limited amount toxic lipid may be presented to the cells during transfection. Thus, when previous methods of forming the lipid/DNA complexes were used, the toxicity of the lipid generally limited the amount of DNA that could be presented to the target cells. Correspondingly, the low amount amounts of DNA that could be offered to cells further limited the transfection efficiencies of lipid-based DNA delivery systems.

6.6 Isolated stable cationic lipid/DNA complexes display low toxicity.

Although the concentrated complexes comprised a many-fold increase in the amount of DNA, they did not display a corresponding increase in toxicity. In fact, concentrated complex made at lipid/DNA phosphate ratio of 15.625 yielded four-fold greater transfection activity than the concentrated and isolated complexes made at a DOSPA/DNA ratio of 6.25, with little to no cellular toxicity observed. Conversely, unconcentrated complex made at the 15.625 ratio is functionally inactive for cellular transfection because of the high levels of cellular toxicity associated with the increased concentration of lipid.

In general, the viability of cells treated with either the transient or stable complexes decreased as the concentration of DNA added to the cells was increased up to 1 μg/ml where essentially all of the treated cells were killed. Conversely, the isolated and resuspended complex caused little toxicity when used to deliver DNA at 1 μg/ml, and there was only minor toxicity at a DNA concentration of 10 μg/ml.

Clearly, by isolating and concentrating the stable complexes one enhances transfection efficiency by both increasing the net amount of DNA that can be presented to cells, and separating the active complex from the major source of cellular toxicity. Subsequent toxicity profiles for the supernatant and the concentrated complex revealed that the vast majority of the toxicity remained in the supernatant.

Additionally, when cellular toxicity studies were conducted that compared the LD₅₀ of various fractions, it was found that the projected lethal dose for the isolated complex (e.g., pellet) was several orders of magnitude higher than either the starting mixture or the supernatant.

When the ratio of lipid to DNA in the concentrated complex was titrated, the percentage of lipid in the complex did not substantially increase after the lipid to DNA ratio was increased to about 10 or higher. Accordingly, one may conclude that the 7.3 kb plasmid used in the study became saturated with associated lipid at a lipid/DNA ratio of about 10. The titration data may prove generically applicable to other nucleic acids, and consideration of such data may further facilitate the disclosed methods of substantially detoxifying lipid/DNA complexes.

6.7 Use of polynucleotide delivery vehicles in vivo.

SPDV were formulated in the presence of 5 mole percent lactosylcerebroside and intravenously injected in to mice. Forty eight hours after injection, whole cellular DNA was extracted from a variety of tissues and Southern analysis was used to screen for plasmid DNA which had been delivered to the host tissue. The data reveal that polynucleotides complexed with the SPDVs were delivered to cells, and that the amount of DNA detected increased as the ratio of DOSPA/DNA phosphate was increased (up to 20:1).

Additionally, SPDVs comprising DNA encoding betagalactosidase were injected into the brain of a live mouse. Subsequent histological studies conducted 72 hrs after injection detected betagalactosidase activity along the needle tract (there was no betagalactosidase activity detected along the needle tract of the control injection). These data are consistent with conclusion that the SPDVs indeed delivered complexed DNA to brain tissue which was subsequently expressed in vivo.

6.8 Comparison of DNA transfer activity of the stable and transient lipid/DNA complex.

Stable and transient complexes were formed essentially as described above at DOSPA to DNA phosphate (mol/mol) ratios varying from 0.3 to 15, and at varying DNA concentrations of 0.1 μg/well (FIG. 11A and 11E), 0.2 μg/well (FIG. 11B and 11F), 0.4 μg/well (FIG. 11C and 11G), and 0.8 μg/well (FIG. 11D and 11H). β-galactosidase activity (solid circles) was plotted on the left y-axis, and compared to total cell protein recovered from cell lysates which was plotted on the right y-axis (open circles).

Among other things, FIG. 11 shows that β-gal expression generally increased as the DNA concentrations were increased for both the transient complex and the stable complex.

6.9 In vitro targeting of stable cationic lipid/DNA delivery vehicles to human endothelial cells using a peptide ligand.

The targeting ligand was a cyclic RGD peptide with the following sequence:

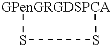

This RGD peptide specifically binds to the α_{υ}β_{δ} integrin receptor. This integrin is expressed on endothelial cells and is able to bind vitronectin. Human umbilical vein endothelial cells (HUVEC) were used as the in vitro tissue culture assay system. Not only do these cells express the vitronectin receptor, but they are very resistant to common DNA transfection techniques. Hence, expression due to non-specific targeting is low thus increasing the signal to noise ratio. The DNA transfection vehicle was assembled as follows.

The strategy for derivatizing SV101 with a targeting ligand first requires that the ligand be attached to a lipid and then incorporated into the detergent/cationic lipid mixed micelle prior to addition of DNA. To accomplish this, the terminal carboxyl group of N-Glutaryldioleoylphosphatidyl-ethanolamine (G-DOPE), purchased from Avanti Polar Lipid, Birmingham, Ala., was converted to a N-hydroxysuccinimide activated ester by reacting the lipid with at molar excess of 1,ethyl-3-(3-dimethylaminopropyl)carbodiimide (EDC) and N-hydroxysuccinimide (NHS). This reaction was carried out by first adding 10 μmol of G-DOPE to a test tube in CHCl₃. The solvent was removed by evaporation under N₂ forming a lipid film and the residual solvent was removed by desiccation. The lipid film was hydrated in water, vortexed into suspension, and sonicated in a strong bath sonicator for 10 minutes. Five fold excess of EDC and NHS was added to the liposomes in 50 mM MES, pH 6.0. The reaction was incubated for 20 minutes at room temperature. The reaction was applied to a 5 ml Sephadex G-25 spin column equilibrated with 10 mM Hepes, pH 8.5 and centrifuged at 2,000 RPM for 3 minutes. The NHS-G-DOPE was collected in the eluate and two micromoles of lipid was solubilized in 1.0 percent octylglucoside. A five fold molar excess of the RGD peptide was added to the solubilized lipid and the reaction was incubated overnight at 4° C.

The lipid/ligand reaction mixture was added to Lipofectamine, purchased from LTI, Gaithersburg, Md., at 5, 10 and 20 mole percent of the total lipid. Octylglucoside was added to make the final octylglucoside concentration one percent. DNA was added to the solubilized lipid mixture at a DOSPA/DNA phosphate ratios of 1, 2.5, 5, 10, 15, and 20. Lipid/DNA mixtures were placed in a 28 well GIBCO dialysis flow chamber and dialyzed against 4 liters of 5 percent dextrose, 10 mM Tris, pH 7.2 for 48 hours.

The HUVECs were plated at a density of 1.5×10⁵ cells per wall in a 35 mm dish 24 hours prior to the addition of DNA. 1 μg of DNA was added to each well in serum free media and allowed to incubate with the cells for 4 hours at 37° C. The lipid/DNA complexes were removed by aspiration and replaced with complete media. The cells were harvested 24 hours later by adding 100 μl of lysis buffer (0.1% Triton X-100 and 250 mM Tris, pH 8.0). Fifty microliters of cell lysate was mixed with 50 μl of S-galactosidase substrate (ONPG) and assayed for beta-galactosidase activity. The results are shown in FIG. 12.

The Y axis of FIG. 12 shows the level of expression observed (as measured by β-galactosidase activity), and the X axis shows the spermine to DNA ratio. DOSPA is composed of diacylglycerol derivatized to spermine. Hence, the spermine to DNA phosphate ratio represents the DOSPA to DNA phosphate ratio. Each line represents an increasing mole percent of the lipid derivatized peptide (lipid-associated ligand). The open circles are the transient complex formed by adding DNA to Lipofectamine in serum free media followed by the immediate application of the transient complex to the cells. No β-galactosidase activity was observed at any of the spermine to DNA phosphate ratios tested.

The closed diamonds represent the stable cationic lipid/DNA complex that showed some gene expression at a ratio of 10 but the level of expression was low, slightly less than 2 milliunits. The five and ten mole percent (targeting peptide-associated lipid/total lipid) are represented by the closed inverted triangle and the closed box, respectively. Both gave the same level of expression at the same spermine/phosphate ratio, 10, which was two fold higher than the non-peptide formulation. The 20 mole percent graph is represented by the upright closed triangle. This formulation gave the maximal level of gene expression which was 10 milliunits at a spermine/phosphate ratio of 10. The results clearly show a strong correlation between the increased level of gene expression and the increasing mole percent of the lipid-associated ligand in the complex which indicate that effective targeting had been achieved.

6.10 Targeting studies using lactosyl cerebroside and fetuin

As discussed above, the stability inherent in the presently disclosed lipid/DNA complexes allows for the manipulations which are often required in order to associate targeting agents to the complexes. For example, stable complexes were formed comprising 10 mole percent of lactosyl cerebroside. This resulted complexes which display galactose residues. The presence of galactose on the surface of the complex is desirable where one wishes to target liver cells (hepatocytes express a surface receptor for galactose).

Alternatively, the protein fetuin may be used. After treatment with neuraminidase (which cleaves sialic acid), asialofetuin results). Asialofetuin is known to associate with the hepatocyte galactose receptor. Coincubation of the stable complex with asialofetuin (precipitated and concentrated stable complex) comprising 0.2 μg DNA with 125 μg of asialofetuin in 0.5 ml for 18 hrs at 4° C.) increased hepatocyte transfection by 15-fold relative to stable complexes which were not preincubated with asialofetuin.

6.11 In vivo expression of genes transfected with concentrated lipid/DNA complex

Concentrated and detoxified stable lipid/DNA complexes containing about 10 μg of DNA were used to introduce the beta-galactosidase gene into mice in vivo. After I.V. (or I.M. injection) administration of the complexes, the mice remained viable and apparently healthy up to the time that they were sacrificed and analyzed for in vivo gene expression. Conversely, mice injected with 10 μg of DNA in the transient complex generally suffer grave damage to their internal organs and rarely survive.

6.12 Alternative method for forming stable SPDVs of reduced toxicity

The plasmid SSV9-MD2-APM (see FIG. 13) was complexed with DOSPA/DOPE (1:1 mol/mol) in a 2 ml volume containing 0.5 mg/ml of DNA, 1 M MgCl₂, and 2% octylglucoside. Cationic lipid (DOSPA) was added at a DOSPA:DNA phosphate ratios of 0.6:1, 1:1, and 1.6:1. The detergent was removed by dialysis at 4° C. into a total of 8 liters of 1 molar MnCl₂ over a period of 48 hours. The subsequent and substantial removal of cation was achieved by dialysis into a total of 8 liters of 5 percent dextrose, 10 mM Tris which resulted in a turbid dispersion of particles with mean diameters between about 50 and about 150 nm. The resulting stable complexes retain full transfection activity for at least two weeks when held at 4° C.

6.13 In vivo expression studies using the stable SPDVs of reduced toxicity

80 μg of DNA (SSV9-pMD-AP) was used to form SPDVs essentially as described in Section 6.12 was injected in a volume of 0.25 ml in the tail vein of a Balb C mouse (approx. 25 gm). MnSV101 refers to MnCl₂ dialyzed lipid/DNA complex. The complexes were prepared with SSV9-pMD-AP that had been prepared by standard plasmid isolation procedures and double banded on CsCl. The DOSPA to DNA ratios varied from about 0.66 to about 1.65. Five mice were injected per group. Tissue samples were harvested 24 hours after administration and homogenized in buffer at a net concentration of 100 mg/ml. The homogenates were heated to 65° C. for 30 minutes to inactivate endogenous alkaline phosphatase. The homogenates were analyzed using an immunocapture assay comprised of adsorbing a secondary antibody to a 96 well plate followed by addition of a anti-human placental alkaline phosphatase polyclonal antibody. 0.2 ml of homogenate was added to each well and allowed to incubate overnight at 4° C. Also included is a standard curve ranging from 20 mUnits to 0.1 mUnits of alkaline phosphatase (AP). The plate was washed and additional 200 ul aliquots were incubated in the wells for 2 hours to increase the signal, or the wells were washed and alkaline phosphatase substrate is added. The plate was read using a Molecular Devices plate reader which can determine a Vₘₐₓ for each well. The Vₘₐₓ was converted to mUnits of AP and the data were normalized per 100 mg of tissue.

FIG. 14 shows that MnSV101 produced significant levels of expression in vivo in all tissues tested except for blood (i.e., liver, spleen, lung, heart, and kidneys). Although MNSV101 prepared at a DOSPA/DNA phosphate ratio of 1.65:1 gave the highest levels of expression, toxicity effects indicate that the ideal ratio probably falls between 1:1 and 1.65:1.

FIG. 15 shows that, for MnSV101 prepared at a DOSPA:DNA phosphate ratio of 1:1, the tested 80 ug dose of DNA generally provided better in vivo expression than lesser concentrations of DNA.

FIG. 16 shows that gene transfer using MnSV101 (produced at a DOSPA:DNA phosphate ratio of 1:1) apparently leads to transient in vivo expression of the delivered DNA.

FIG. 17 shows that stable synthetic delivery vehicles produced essentially as described in Section 6.12 (at a DOSPA:DNA phosphate ratio of 1:1) may also be produced using the monovalent cation Na (using NaCl, i.e., NaSV101, or NaCl-SV101) at concentrations similar to those used when the divalent Mn cation is used. The data in FIG. 17 further indicate that simply reducing, as opposed to substantially removing, the concentration of cation in the complex during the second dialysis step may yield enhanced levels of expression when sodium is used to help form SPDVs. FIG. 17 also shows that MnSV101 and NaSVO101 are both capable of delivering a gene to muscle tissue which can subsequently express the gene in vivo.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

1. A stable synthetic polynucleotide delivery vehicle comprising an amphipathic cationic lipid conjugate.

2. A stable synthetic polynucleotide delivery vehicle comprising a biodegradable amphipathic cationic lipid conjugate.

3. A delivery vehicle according to claim 1, which further comprises a targeting ligand.

4. A delivery vehicle according to claim 2, which further comprises a targeting ligand.

5. A delivery vehicle according to claims 1, 2, 3, or 4 which is stable in serum.

6. A delivery vehicle according to claim 5 which is size stable.

7. A process for making a stable polynucleotide delivery vehicle, comprising:
a) contacting the polynucleotide with a amphipathic cationic lipid conjugate in the presence of a detergent; and
b) removing the detergent to form a polynucleotide and cationic lipid complex.

8. A stable polynucleotide delivery vehicle produced by the method of claim 7.

9. The stable polynucleotide delivery vehicle of claim 8 which comprises a biodegradable amphipathic cationic lipid conjugate.

10. The stable polynucleotide delivery vehicle of claim 8 or 9 which comprises a targeting ligand.

11. The stable polynucleotide delivery vehicle of claim 10 which is serum stable.

12. A biocompatible amphipathic cationic lipid conjugate having the general formula:
R₁-X-R₂
wherein R₁ is a biodegradable lipid moiety; R₂ is a biocompatible cationic or polycationic moiety; and
X is a biocompatible and labile covalent linker.

13. The use of a stable polynucleotide delivery vehicle to deliver a polynucleotide of interest to a cell.

14. The use of claim 13 wherein said cell is present in vitro.

15. The use of claim 13 wherein said cell is present in vivo.

16. The use of the biodegradable amphipathic cationic lipid of claim 12 to prepare a lipid formulation.

17. A use according to claim 13 wherein said stable polynucleotide delivery vehicle comprises a biodegradable amphipathic cationic lipid.

18. The use of claim 17 wherein said cell is present in vitro.

19. The use of claim 17 wherein said cell is present in vivo.

20. A stable synthetic polynucleotide delivery vehicle of reduced toxicity.

21. The use of a delivery vehicle according to claim 20 to effect gene transfer into a cell.

22. The use of claim 21 wherein said cell is present in vitro.

23. The use of claim 21 wherein said cell is present in vivo.

24. A method of producing a delivery vehicle according to claim 20 comprising:
a) contacting a polynucleotide with an amphipathic cationic lipid conjugate in the presence of a detergent;
b) removing the detergent to form a polynucleotide and cationic lipid complex; and
c) substantially isolating the complex from unassociated lipid.

25. A process for making a stable polynucleotide delivery vehicle, comprising:
a) contacting the polynucleotide with a amphipathic cationic lipid conjugate in the presence of a cation and detergent; and
b) removing the detergent to form a polynucleotide and cationic lipid complex.

26. A method according to claim 25 wherein said detergent is removed prior to said cation.

27. A method according to claim 25 where said cation is present at a concentration of at least about 0.1 molar.

28. A method according to claim 25 where said cation is substantially removed after the detergent is removed.
